(19) United States
(12) Patent Application Publication
(10) Pub. No.: US 2024/0415855 A1
(43) Pub. Date: Dec. 19, 2024

(54) COMPOSITIONS AND METHODS RELATING TO USE OF PHOSPHATIDIC ACID TO RESCUE FETAL ALCOHOL SPECTRUM DISORDER (FASD) GROWTH RESTRICTION AND BLOOD VESSEL CONSTRICTION

(71) Applicant: Wayne State University, Detroit, MI (US)

(72) Inventors: Jayanth Ramadoss, Detroit, MI (US); Vishal Naik, Detroit, MI (US)

(21) Appl. No.: 18/742,267

(22) Filed: Jun. 13, 2024

Related U.S. Application Data
(60) Provisional application No. 63/472,975, filed on Jun. 14, 2023.

(51) Int. Cl. A61K 31/661; A61P 3/00; A61P 25/00; A61P 43/00

(52) U.S. Cl. CPC A61K 31/661; A61P 3/00; A61P 25/00; A61P 43/00

(57) ABSTRACT

Methods of ameliorating one or more effects of exposure to ethanol in a pregnant subject, and/or a fetal subject in utero, and/or in a post-natal subject, who was exposed to ethanol in utero, are provided according to aspects of the present disclosure, which include: administering, to the pregnant subject, and/or the fetal subject, and/or post-natal subject, a therapeutically effective amount of a lipid which is downregulated in a pregnant subject exposed to ethanol and/or a fetus exposed to ethanol in utero. Pharmaceutical compositions are provided according to aspects of the present disclosure which include a therapeutically effective amount of a lipid which is downregulated in a pregnant subject exposed to ethanol, and/or a fetus, exposed to ethanol in utero; and a pharmaceutically acceptable excipient.

### REFERENCE TO RELATED APPLICATION

This application claims priority to U.S. Provisional Patent Application Ser. No. 63/472,975, filed Jun. 14, 2023, the entire content of which is incorporated herein by reference.

### GOVERNMENT SUPPORT

This invention was made with government support under R01AA023520 awarded by National Institutes for Health. The government has certain rights in the invention.

### FIELD OF THE INVENTION

According to general aspects, the present disclosure relates to compositions and methods for treatment of Fetal Alcohol Spectrum Disorder. According to specific aspects, the present disclosure relates to compositions including one or more lipids and methods including administration of one or more lipids for treatment of Fetal Alcohol Spectrum Disorder.

### BACKGROUND OF THE INVENTION

Gestational use of ethanol is estimated to be ˜9.8% globally, with use during early pregnancy recently estimated to be 49.7% in eight metropolitan areas in the south/south-east regions of the United States and binge alcohol use during pregnancy estimated at 3.1%.

Consuming ethanol during gestation can significantly impact fetal development, leading to Fetal Alcohol Spectrum Disorder (FASD). FASD are conditions that can occur when an individual is exposed to ethanol before birth. FASD is characterized by a spectrum of physical and neurobehavioral effects, which include lower birth weights, shorter-than average heights, and other developmental impairments. Recent estimates range from 3.1 to 9.9% of school-aged children having a FASD.

A cardinal feature of FASD is growth restriction. Growth deficits associated with prenatal alcohol exposure are observed among children from birth until at least 14 years of age. FASD data, spanning from the 1970s to present times, from human children and from animal studies, demonstrate reduced weight, height, and birth head circumference.

Fetal growth, neonatal birth weight, and survival are all directly related to major uterine circulatory adaptations in normal pregnancies. Uterine arteries are unique among all blood vessels during pregnancy as their diameter doubles from nonpregnant state to second trimester and triples by third trimester in humans. These adaptations are accompanied by a change in the blood velocity from 8 cm/s in the nonpregnant luteal state to almost 61 cm/s in the third trimester of gestation. Further, the uterine blood flow increases by ˜25 fold in the third trimester compared to the nonpregnant luteal phase. Gestational uterine vascular adaptations are critical for gas and nutrient delivery and are correlated with fetal growth.

Alcohol is shown to have a direct impact on uterine arterial adaptations, including endothelium-dependent acetylcholine-mediated uterine artery vasodilation in rats and sheep, in addition to vessel remodeling at the level of the spiral artery. In vitro alcohol exposure alters the transcriptome and the proteome of the uterine artery. The most severe of the FASD is Fetal Alcohol Syndrome (FAS), a term that describes specific clinically-diagnosable morphological and functional manifestations, including intrauterine growth restriction (IUGR).

IUGR is directly related to compromised uterine blood flow, increased uterine artery resistance, and arterial remodeling. Total volumetric blood flow through the uterine artery is significantly lower in cases of IUGR, and the primary uterine artery along with its proximal segments exhibit less dilation than in normal pregnancies. The uterine artery is a unique vasculature that undergoes major remodeling during gestation to deliver oxygen and nutrients to the developing fetus. During a normal pregnancy, a significant pathway to induce uterine artery vasorelaxation at least in the primary uterine artery is the endothelial nitric oxide system.

There are currently no known effective treatments for ameliorating one or more effects of exposure to ethanol in a subject while in utero and/or in a post-natal subject who was exposed to ethanol in utero. Thus, there is a continuing need for compositions and methods to ameliorate one or more effects of exposure to ethanol in a subject while in utero and/or in a post-natal subject who was exposed to ethanol in utero.

### SUMMARY OF THE INVENTION

Methods of ameliorating one or more effects of exposure to ethanol in a pregnant subject, and/or a fetal subject in utero, and/or in a post-natal subject, who was exposed to ethanol in utero, are provided according to aspects of the present disclosure, which include: administering, to the pregnant subject, and/or the fetal subject, and/or post-natal subject, a therapeutically effective amount of a lipid which is downregulated in a pregnant subject exposed to ethanol and/or a fetus exposed to ethanol in utero. According to aspects of the present disclosure, the lipid is selected from the group consisting of: a glycerophosphethanolamine, a glycerophosphocholine, a triacylglycerol, a phosphosphingolipid, a glycerophosphoinositol, a glycerophosphoserine; and a combination of any two or more thereof. According to aspects of the present disclosure, the lipid is selected from the group consisting of: a phosphatidic acid, a phosphatidylcholine, a phosphatidylethanolamine, a phosphatidylinositol, a phosphatidylserine; and a combination of any two or more thereof.

Methods of ameliorating one or more effects of exposure to ethanol in a pregnant subject, and/or a fetal subject in utero, and/or in a post-natal subject, who was exposed to ethanol in utero, are provided according to aspects of the present disclosure, which include: administering, to the pregnant subject, and/or the fetal subject, and/or post-natal subject, a therapeutically effective amount of a phosphatidic acid.

Methods of ameliorating one or more effects of exposure to ethanol in a pregnant subject, and/or a fetal subject in utero, and/or in a post-natal subject, who was exposed to ethanol in utero, are provided according to aspects of the present disclosure, which include: administering, to the pregnant subject, and/or the fetal subject, and/or post-natal subject, a therapeutically effective amount of a phosphatidic acid or a mixture of phosphatidic acids, each phosphatidic acid having the chemical structure:

where each R1 and R2 is

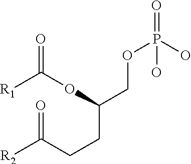

independently selected from saturated or unsaturated C16 to C22 phosphatidic acid.

Pharmaceutical compositions are provided according to aspects of the present disclosure which include a therapeutically effective amount of a lipid which is downregulated in a pregnant subject exposed to ethanol, and/or a fetus, exposed to ethanol in utero; and a pharmaceutically acceptable excipient.

Pharmaceutical compositions are provided according to aspects of the present disclosure which include a therapeutically effective amount of one or more of the following: a glycerophosphethanolamine, a glycerophosphocholine, a triacylglycerol, a phosphosphingolipid, a glycerophosphoinositol, and a glycerophosphoserine.

Pharmaceutical compositions are provided according to aspects of the present disclosure which include a therapeutically effective amount of one or more of the following: a phosphatidic acid, a phosphatidylcholine, a phosphatidylethanolamine, a phosphatidylinositol, and a phosphatidylserine.

Pharmaceutical compositions are provided according to aspects of the present disclosure which include a therapeutically effective amount of a phosphatidic acid.

Pharmaceutical compositions are provided according to aspects of the present disclosure which include a therapeutically effective amount of a phosphatidic acid, or a mixture of phosphatidic acids, each phosphatidic acid having the chemical structure:

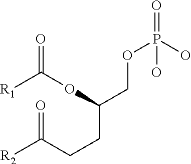

where each R1 and R2 is independently selected from saturated or unsaturated C16 to C22 phosphatidic acid.

Methods of inhibiting and/or reversing growth restriction of a fetus of a subject pregnant with the fetus, wherein the fetus is exposed to ethanol in utero, are provided according to aspects of the present disclosure which include administering, to the pregnant subject, a therapeutically effective amount of a lipid which is downregulated in a pregnant subject exposed to ethanol during pregnancy.

Methods of inhibiting and/or reversing growth restriction of a fetus of a subject pregnant with the fetus, wherein the fetus is exposed to ethanol in utero, are provided according to aspects of the present disclosure which include administering, to the pregnant subject, a therapeutically effective amount of one or more of: a glycerophosphethanolamine, a glycerophosphocholine, a triacylglycerol, a phosphosphingolipid, a glycerophosphoinositol, and a glycerophosphoserine, which is downregulated in a pregnant subject exposed to ethanol during pregnancy.

Methods of inhibiting and/or reversing growth restriction of a fetus of a subject pregnant with the fetus, wherein the fetus is exposed to ethanol in utero, are provided according to aspects of the present disclosure which include administering, to the pregnant subject, a therapeutically effective amount of one or more of: a phosphatidic acid, a phosphatidylcholine, a phosphatidylethanolamine, a phosphatidylinositol, and a phosphatidylserine, which is downregulated in a pregnant subject exposed to ethanol during pregnancy.

Methods of inhibiting and/or reversing growth restriction of a fetus of a subject pregnant with the fetus, wherein the fetus is exposed to ethanol in utero, are provided according to aspects of the present disclosure which include administering, to the pregnant subject, a therapeutically effective amount of a phosphatidic acid which is downregulated in a pregnant subject exposed to ethanol during pregnancy.

Methods of inhibiting and/or reversing growth restriction of a fetus of a subject pregnant with the fetus, wherein the fetus is exposed to ethanol in utero, are provided according to aspects of the present disclosure which include administering, to the pregnant subject, a therapeutically effective amount of a phosphatidic acid, or a mixture of phosphatidic acids, each phosphatidic acid having the chemical structure:

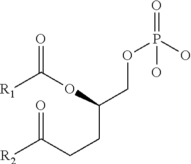

where each R1 and R2 is independently selected from saturated or unsaturated C16 to C22 phosphatidic acid.

Methods of inhibiting and/or reversing blood vessel constriction in a pregnant subject exposed to ethanol during pregnancy are provided according to aspects of the present disclosure which include administering, to the pregnant subject, a therapeutically effective amount of a lipid which is downregulated in a pregnant subject exposed to ethanol during pregnancy.

Methods of inhibiting and/or reversing blood vessel constriction in a pregnant subject exposed to ethanol during pregnancy are provided according to aspects of the present disclosure which include administering, to the pregnant subject, a therapeutically effective amount of one or more of: a glycerophosphethanolamine, a glycerophosphocholine, a triacylglycerol, a phosphosphingolipid, a glycerophosphoinositol, and a glycerophosphoserine, which is downregulated in a pregnant subject exposed to ethanol during pregnancy.

Methods of inhibiting and/or reversing blood vessel constriction in a pregnant subject exposed to ethanol during pregnancy are provided according to aspects of the present disclosure which include administering, to the pregnant subject, a therapeutically effective amount of one or more of: a phosphatidic acid, a phosphatidylcholine, a phosphatidylethanolamine, a phosphatidylinositol, and a phosphatidylserine, which is downregulated in a pregnant subject exposed to ethanol during pregnancy.

Methods of inhibiting and/or reversing blood vessel constriction in a pregnant subject exposed to ethanol during pregnancy are provided according to aspects of the present disclosure which include administering, to the pregnant subject, a therapeutically effective amount of a phosphatidic acid which is downregulated in a pregnant subject exposed to ethanol during pregnancy.

Methods of inhibiting and/or reversing blood vessel constriction in a pregnant subject exposed to ethanol during pregnancy are provided according to aspects of the present disclosure which include administering, to the pregnant subject, a therapeutically effective amount of a phosphatidic acid, or a mixture of phosphatidic acids, wherein each phosphatidic acid has the chemical structure:

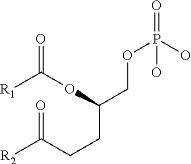

where each R1 and R2 is independently selected from saturated or unsaturated C16 to C22 phosphatidic acid.

### BRIEF DESCRIPTION OF THE DRAWINGS

FIG. 1A is drawing showing the chemical structure of phosphatidinylethanol (PEth) 16:0/18:1; PEth is a group of phospholipids formed in the presence of ethanol, phospholipase D and phosphatidylcholine; PEth is known to be a direct alcohol biomarker;

FIG. 1B is a drawing showing the chemical structure of PEth 16:0/18:2;

FIG. 1C is a drawing showing the chemical structure of PEth 16:0/20:4;

FIG. 2A is a graph showing relative amounts of PEth in blood from maternal (Mat) and fetal (Fet) Pair-Fed (PF)-Control or Ethanol (Alcohol)-exposed subjects determined by Mass Spectrometry;

FIG. 2B is a graph showing relative amounts of PEth in uterine artery from maternal PF-Control or Ethanol (Alcohol)-exposed subjects determined by Mass Spectrometry;

FIG. 2C is a graph showing relative amounts of PEth in fetal brain from fetal PF-Control or Ethanol (Alcohol)-exposed determined by Mass Spectrometry;

FIG. 3 is a diagram illustrating a process for analysis of various lipid species by Mass Spectrometry;

FIG. 4A is a graph showing relative amounts of various lipid species measured in Pair-Fed Controls (light bars) or Ethanol-fed (Alcohol) subjects (black bars);

FIG. 4B is a graph showing relative amounts of various lipid species measured in Pair-Fed Controls (light bars) or Ethanol-fed (Alcohol) subjects (black bars);

FIG. 4C is a graph showing relative amounts of various lipid species measured in Pair-Fed Controls (light bars) or Ethanol-fed (Alcohol) subjects (black bars);

FIG. 4D is a graph showing relative amounts of various lipid species measured in Pair-Fed Controls (light bars) or Ethanol-fed (Alcohol) subjects (black bars);

FIG. 5A is a graph showing relative amounts of various lipid species measured in Pair-Fed Controls (light bars) or Ethanol-fed (Alcohol) subjects (black bars);

FIG. 5B is a graph showing relative amounts of various lipid species measured in Pair-Fed Controls (light bars) or Ethanol-fed (Alcohol) subjects (black bars);

FIG. 6A is a graph showing relative amounts of various lipid species measured in Pair-Fed Controls (light bars) or Ethanol-fed (Alcohol) subjects (black bars);

FIG. 6B is a graph showing relative amounts of various lipid species measured in Pair-Fed Controls (light bars) or Ethanol-fed (Alcohol) subjects (black bars);

FIG. 7A is a graph showing relative amounts of various lipid species measured in Pair-Fed Controls (light bars) or Ethanol-fed (Alcohol) subjects (black bars);

FIG. 7B is a graph showing relative amounts of various lipid species measured in Pair-Fed Controls (light bars) or Ethanol-fed (Alcohol) subjects (black bars);

FIG. 8A is a table summarizing lipid subclasses affected by ethanol;

FIG. 8B is a graph summarizing lipid subspecies altered by ethanol;

FIG. 9 is a diagram showing conversion of phosphatidyl choline by phospholipase D to phosphatidic acid in the presence of water or to phosphatidylethanol in the presence of ethanol, each R1 and R2 in the structure shown for phosphatidic acid is a fatty acid having a number of carbon atoms independently selected from 16 to 22;

FIG. 10A is a graph showing PE % Max as a function of PE in control and ethanol-exposed subjects;

FIG. 10B is a graph showing % KCL as a function of PE in control and ethanol-exposed subjects;

FIG. 11A is a graph showing TBX % Max as a function of TBX in control and ethanol-exposed subjects;

FIG. 11B is a graph showing % KCL as a function of TBX in control and ethanol-exposed subjects;

FIG. 12A is a graph showing % Relaxation as a function of SNP in control and ethanol-exposed subjects;

FIG. 12B is a graph showing % Relaxation as a function of Acetylcholine (ACH) in control and ethanol-exposed subjects;

FIG. 13A is a graph showing relative levels of Ach pD2 in control and ethanol-exposed subjects;

FIG. 13B is a graph showing Ach EMax in control and ethanol-exposed subjects;

FIG. 14A is an image showing results of 2D-DIGE proteomics in uterine artery of control subjects;

FIG. 14B is an image showing results of 2D-DIGE proteomics in uterine artery of ethanol-exposed subjects;

FIG. 14C is a merged image of FIG. 14A and FIG. 14B showing changes in proteins in uterine artery of control and ethanol-exposed subjects;

FIG. 15A is an image showing results of 2D-DIGE proteomics in uterine artery of control subjects;

FIG. 15B is an image showing results of 2D-DIGE proteomics in uterine artery of ethanol-exposed subjects;

FIG. 15C is a merged image of FIG. 15A and FIG. 15B showing changes in proteins in uterine artery of control and ethanol-exposed subjects;

FIG. 15D is an image showing results of 2D-DIGE proteomics in uterine artery of control subjects;

FIG. 15E is an image showing results of 2D-DIGE proteomics in uterine artery of ethanol-exposed subjects;

FIG. 15F is a graph showing changes in proteins in uterine artery of control and ethanol-exposed subjects;

FIG. 16 is a set of images and a graph showing changes in levels of Annexin A2 and Annexin A2 Chain A by 2D-DIGE proteomics and immunoblot in uterine artery of control and ethanol-exposed subjects;

FIG. 17 is a heat-map diagram showing results of mass spectrometry analysis of proteins in uterine artery of control and ethanol-exposed subjects;

FIG. 18 is a graph illustrating aspects of the citrulline/nitric oxide pathway;

FIG. 19 is a diagram illustrating aspects of the citrulline/nitric oxide pathway;

FIG. 20 is a graph showing results of a functional assay of uterine artery under ex vivo pressure conditions in tissue excised from PF-control subjects and ethanol-exposed subjects;

FIG. 21 is a set of images of immunoblots and graphs derived from the immunoblot data showing levels of P-Ser1177 eNOS, total eNOS, and beta-actin in PF-Control, Control, and ethanol-exposed uterine artery;

FIG. 22A is a graph showing effects of administration of phosphatidic acid (PA) in varying concentrations on vessel diameter in uterine artery tissue ex vivo from PF-Control (PF-Cont), and ethanol-exposed (Alcohol) subjects;

FIG. 22B is a graph showing effects of administration of phosphatidic acid (PA) in varying concentrations on vessel % relaxation in uterine artery tissue ex vivo from PF-Control (PF-Cont), and ethanol-exposed (Alcohol) subjects;

FIG. 23A is a graph showing effects of administration of 10⁻⁵ M Ach on vessel % relaxation in uterine artery tissue ex vivo from PF-Control (PF-Cont), and ethanol-exposed (Alcohol) subjects;

FIG. 23B is a graph showing effects of administration of 10⁻⁵ M Ach and 10⁻⁵ M phosphatidic acid (PA) on vessel % relaxation in uterine artery tissue ex vivo from PF-Control (PF-Cont), and ethanol-exposed (Alcohol) subjects;

FIG. 24A is a set of images of immunoblots and a graph derived from the immunoblot data showing effects of administration of phosphatidic acid (PA) on levels of P-Ser1177 eNOS/total eNOS in PF-Control, and ethanol-exposed (Alcohol) uterine artery;

FIG. 24B is a set of images of immunoblots and a graph derived from the immunoblot data showing effects of administration of phosphatidic acid (PA) on levels of P-total eNOS/GAPDH in PF-Control, and ethanol-exposed (Alcohol) uterine artery;

FIG. 25 is a diagram illustrating a beneficial effect of phosphatidic acid on ethanol-exposed maternal uterine artery functional characteristics;

FIG. 26A is a graph showing that maternal body weights did not differ among the treatment groups: Control, Ethanol (Alcohol), a control substance and phosphatidic acid (Control PA), or ethanol and phosphatidic acid (Alcohol PA);

FIG. 26B is a graph showing that mean fetal weights were significantly lower in the alcohol group compared with those in the control group (p<0.0001), however, the fetal weight difference between the alcohol group and the pair-fed control group was completely abolished by concomitant in vivo PA administration with alcohol, indicating reversal of classic FASD growth restriction phenotype;

FIG. 26C is an image showing representative fetuses that are in the median weight range in each of the four treatment groups: control substance (CTRL), ethanol (ALC), a control substance and phosphatidic acid (CPA), or ethanol and phosphatidic acid (APA);

FIG. 26D is a graph showing the effect of ethanol (Alcohol) on total maternal plasma phosphatidic acid level;

FIG. 26E is a graph showing the effect of ethanol (Alcohol) on total maternal plasma phosphatidyl ethanol level;

FIG. 27A is a graph showing the percent relaxation in response to various amounts of acetylcholine (Ach) of rat uterine arteries isolated from pregnant rat subjects following in vivo administration of a control substance (Control), ethanol (Alcohol), a control substance and phosphatidic acid (Control+PA), or ethanol and phosphatidic acid (Alcohol+PA);

FIG. 27B is a graph showing the percent relaxation in response to various amounts of acetylcholine of rat uterine arteries isolated from pregnant rat subjects following in vivo administration of a control substance (Control), or ethanol (Alcohol);

FIG. 27C is a graph showing the percent relaxation in response to various amounts of acetylcholine of rat uterine arteries isolated from pregnant rat subjects following in vivo administration of ethanol (Alcohol) or ethanol and phosphatidic acid (Alcohol+PA);

FIG. 27D is a graph showing the maximal percent relaxation in response to 3e⁻⁶ acetylcholine of rat uterine arteries isolated from pregnant rat subjects following in vivo administration of a control substance (Control), or ethanol (Alcohol), with or without administration of phosphatidic acid (PA);

FIG. 28 is a set of images showing effects of in vivo administration of a control substance (Control), ethanol (Alcohol), a control substance and phosphatidic acid (Control+PA), or ethanol and phosphatidic acid (Alcohol+PA) to pregnant subjects;

FIG. 29 is a graph showing ethanol-induced decreases in stimulatory p1177-eNOS phosphorylation in the endothelium of the uterine artery; significance (**) was established a priori at p<0.05;

FIG. 30 is a graph showing that in vivo Phosphatidic acid (PA) reverses alcohol-induced decreases in the uterine artery excitatory p1177-eNOS levels in pregnant rats; significance as established a priori at p<0.05; ns, not significantly different;

FIG. 31 is a graph showing the effect of ethanol (Alcohol) and/or in vivo PA on uterine artery total eNOS expression and its localization; significance was established a priori at p<0.05. ns, not significantly different;

FIG. 32 is a diagrammatic representation of a mechanistic model of interaction between in vivo PA and alcohol in rat uterine artery; and

FIG. 33 is a drawing showing phosphatidic acid wherein each R1 and R2 in the structure is a saturated or unsaturated fatty acid having a number of carbon atoms independently selected from C16 to C22, e.g. 16:0, 16:1, 16:2, 18:0, 18:1, 18:2, 18:3, 18:4, 20:1, 20:2, 20:3, 20:4, 20:5, 20:6, 22:1, 22:2, 22:3, 22:4, 22:5, 22:6.

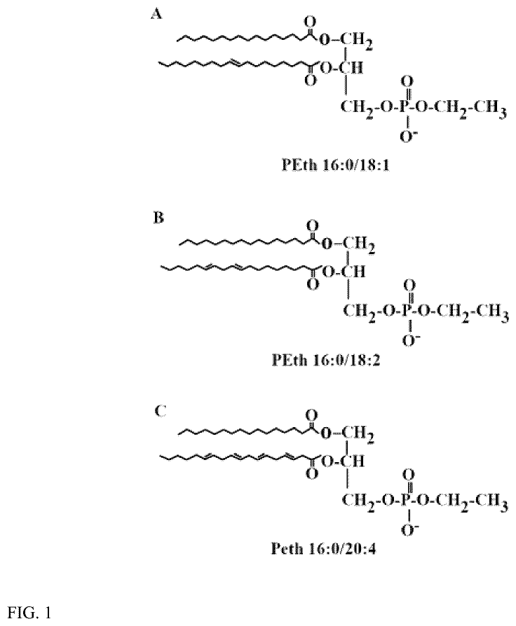

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; CRISPR/Cas: A Laboratory Manual, Doudna and Mali (eds), Cold Spring Harbor Laboratory Press, Cold Spring Harbor, NY, USA, 2016; D. L. Nelson and M. M. Cox, Lehninger Principles of Biochemistry, 4th Ed., W.H. Freeman & Company, 2004; J.-H. Fuhrhop et al. (Eds.), Organic Synthesis, Concepts and Methods, 3rd Ed., Wiley-VCH Cerlag GmbH & Co. KGaA, 2003; Herdewijn, P. (Ed.), Oligonucleotide Synthesis: Methods and Applications, Methods in Molecular Biology, Humana Press, 2004; D. J. Taxman (ed.), siRNA Design, Methods and Protocols, Humana Press, 2012; Harlow, E. and Lane, D., Antibodies: A Laboratory Manual, Cold Spring Harbor Laboratory Press, 1988; J. D. Pound (Ed.) Immunochemical Protocols, Methods in Molecular Biology, Humana Press, 2nd ed., 1998; Chu, E. and Devita, V. T., Eds., Physicians' Cancer Chemotherapy Drug Manual, Jones & Bartlett Publishers, 2021; J. M. Kirkwood et al., Eds., Current Cancer Therapeutics, 4th Ed., Current Medicine Group, 2001; A Adejare (Ed.), Remington: The Science and Practice of Pharmacy, Elsevier, 23rd Ed., 2021; L. V. Allen, Jr. et al., Ansel's Pharmaceutical Dosage Forms and Drug Delivery Systems, 11th Ed., Wolters Kluwer, 2016; and L. Brunton et al., Goodman & Gilman's The Pharmacological Basis of Therapeutics, McGraw-Hill Education, 13th Ed., 2018.

The singular terms “a,” “an,” and “the” are not intended to be limiting and include plural referents unless explicitly stated otherwise or the context clearly indicates otherwise.

The terms “includes,” “comprises,” “including,” “comprising,” “has,” “having,” and grammatical variations thereof, when used in this specification, are not intended to be limiting, and specify the presence of stated features, elements, and/or components, but do not preclude the presence or addition of one or more other features, elements, components, and/or groups thereof.

The term “about” as used herein in reference to a number is used herein to include numbers which are greater, or less than, a stated or implied value by 1%, 5%, 10%, or 20%.

Particular combinations of features are recited in the claims and/or disclosed in the specification, and these combinations of features are not intended to limit the disclosure of various aspects. Combinations of such features not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a alone; b alone; c alone, a and b, a, b, and c, b and c, a and c, as well as any combination with multiples of the same element, such as a and a; a, a, and a; a, a, and b; a, a, and c; a, b, and b; a, c, and c; and any other combination or ordering of a, b, and c).

The terms “first,” “second,” and the like are used herein to describe various features or elements, but these features or elements are not intended to be limited by these terms, but are only used to distinguish one feature or element from another feature or element. Thus, a first feature or element could be termed a second feature or element, and vice versa, without departing from the teachings of the present disclosure.

Methods and compositions of the present disclosure are useful in amelioration of signs and/or symptoms of exposure to ethanol in a pregnant subject and/or a fetal subject in utero and/or in a post-natal subject who was exposed to ethanol in utero. The terms “ameliorate”, “amelioration”, and grammatical equivalents, are used to refer to inhibiting or reducing one or more signs and/or symptoms of exposure to ethanol in a pregnant subject, and/or a fetal subject in utero, and/or in a post-natal subject, who was exposed to ethanol in utero, such as inhibiting or reducing one or more signs and/or symptoms of Fetal Alcohol Spectrum Disorders (FASD). Non-limiting examples of signs and/or symptoms of FASD include low body weight. According to aspects of the present disclosure, the term “ameliorate” refers to increasing levels of a lipid which is downregulated in a pregnant subject and/or a fetal subject in utero and/or in a post-natal subject by exposure of the pregnant subject to ethanol, thereby exposing her fetus to ethanol in utero.

Methods of ameliorating one or more effects of exposure to ethanol in a pregnant subject, and/or a fetal subject in utero, and/or in a post-natal subject, who was exposed to ethanol in utero, are provided according to aspects of the present disclosure which include: administering, to the pregnant subject, and/or the fetal subject, and/or post-natal subject, a therapeutically effective amount of a lipid which is downregulated in a pregnant subject exposed to ethanol and/or a fetus exposed to ethanol in utero.

According to aspects of the present disclosure, the lipid is selected from the group consisting of: a glycerophosphethanolamine, a glycerophosphocholine, a triacylglycerol, a phosphosphingolipid, a glycerophosphoinositol, and a glycerophosphoserine.

According to aspects of the present disclosure, the lipid is selected from the group consisting of: a phosphatidic acid, a phosphatidylcholine, a phosphatidylethanolamine, a phosphatidylinositol, and a phosphatidylserine.

According to aspects of the present disclosure, the lipid is a phosphatidic acid.

Methods of ameliorating one or more effects of exposure to ethanol in a pregnant subject, and/or a fetal subject in utero, and/or in a post-natal subject, who was exposed to ethanol in utero, are provided according to aspects of the present disclosure which include: administering, to the pregnant subject, and/or the fetal subject, and/or post-natal subject, a therapeutically effective amount of a phosphatidic acid which is downregulated in a pregnant subject exposed to ethanol and/or a fetus exposed to ethanol in utero.

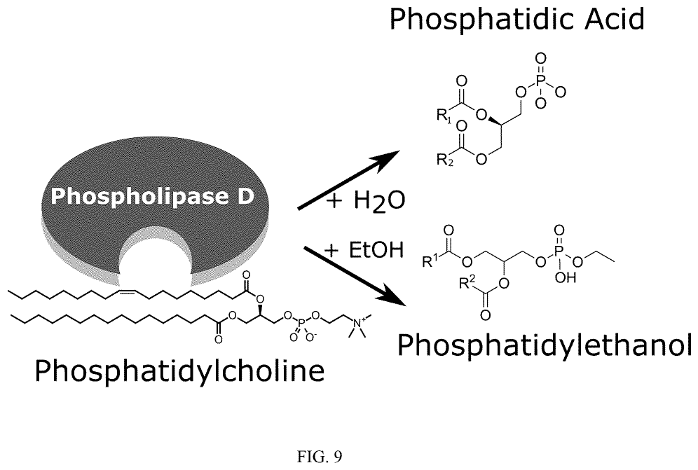

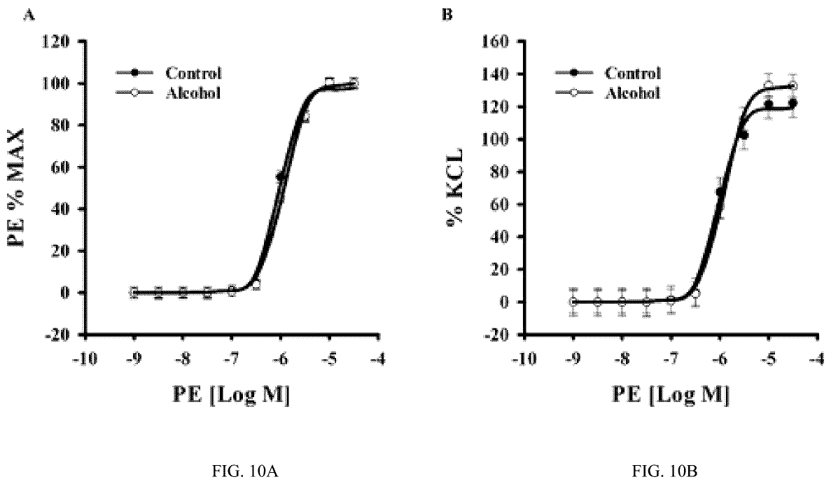

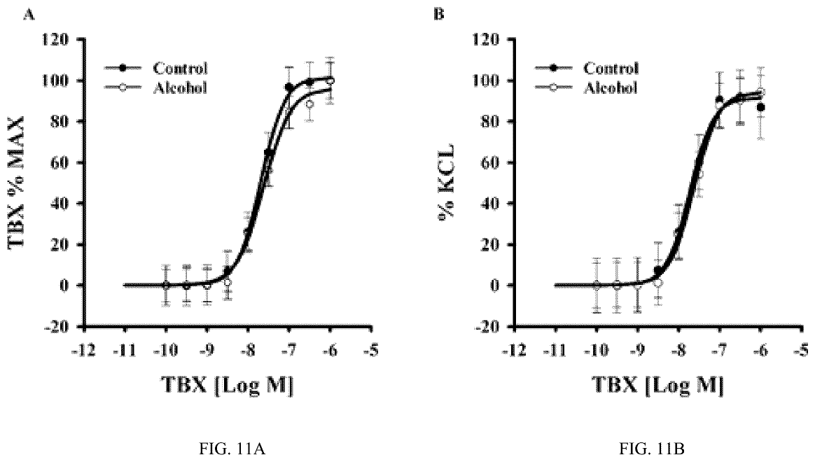

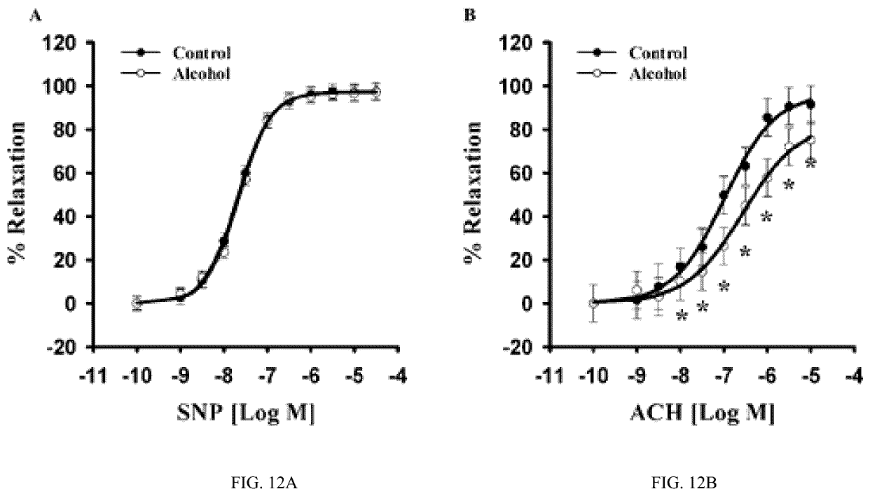

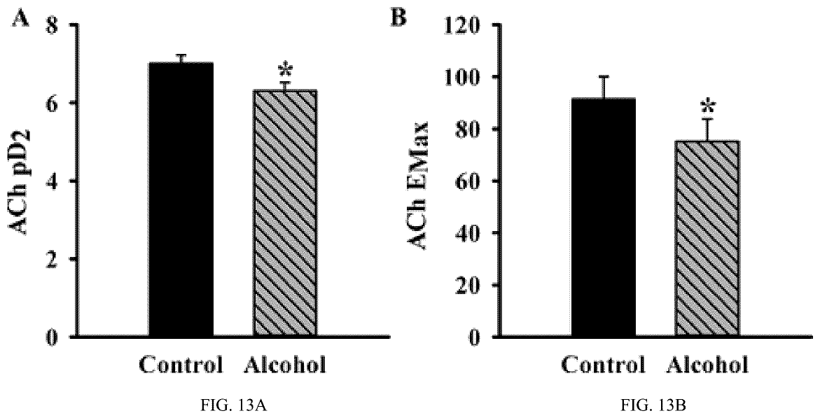

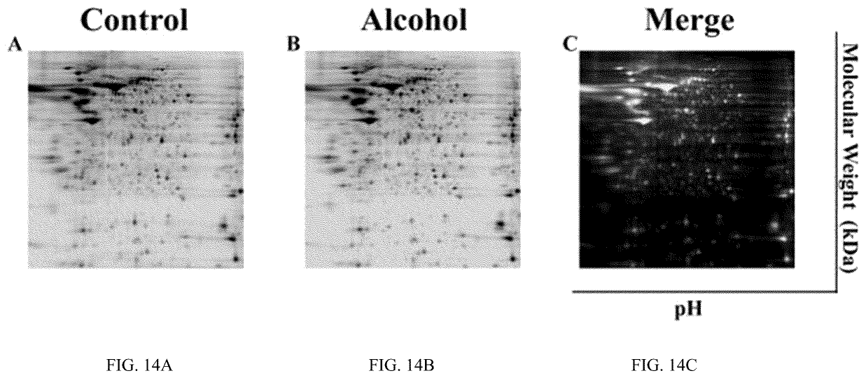

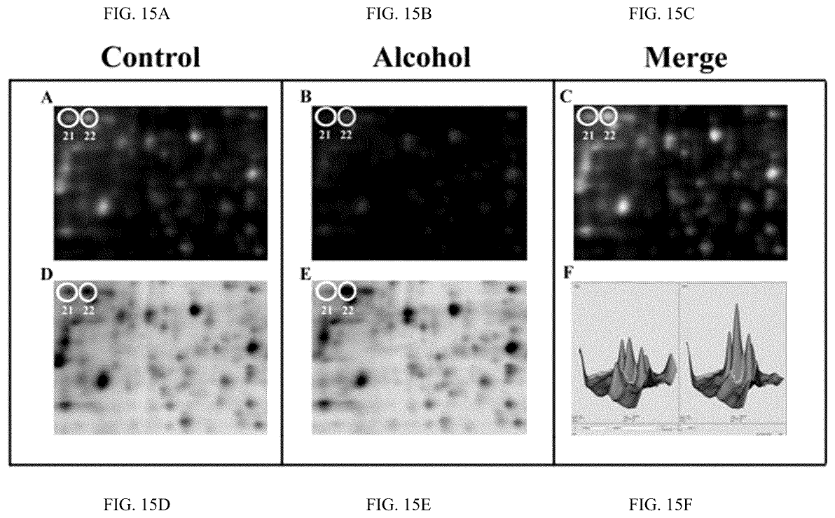

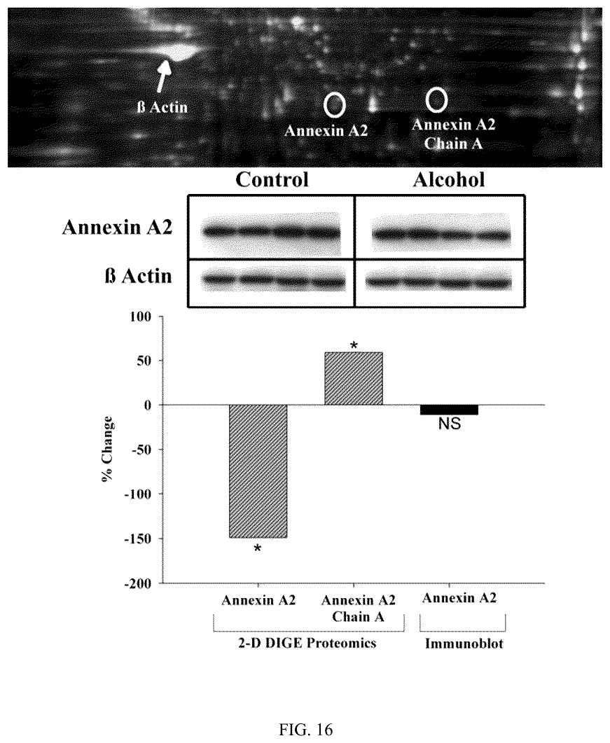

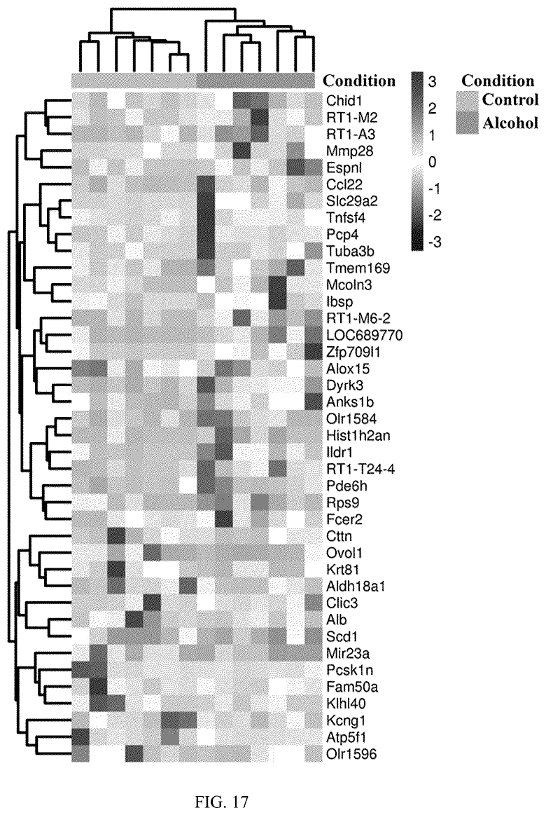

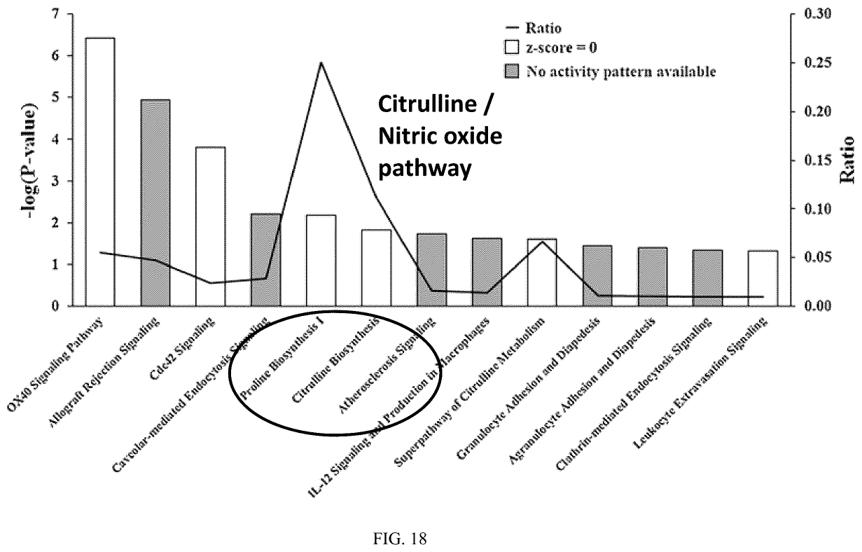

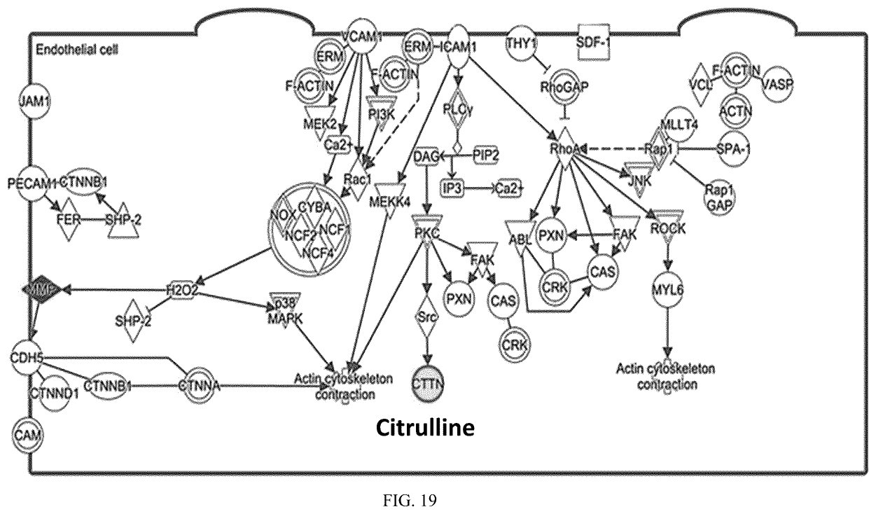

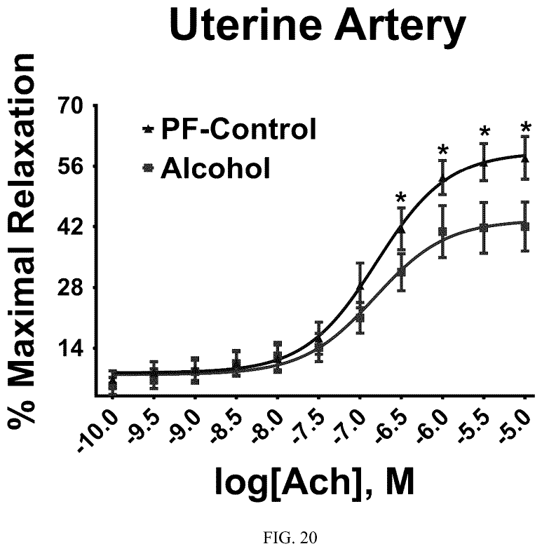

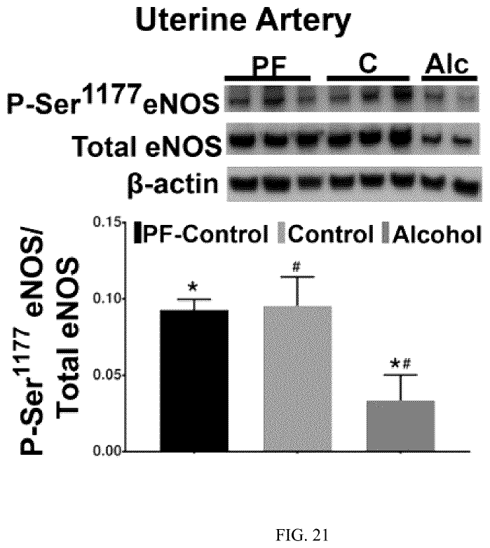

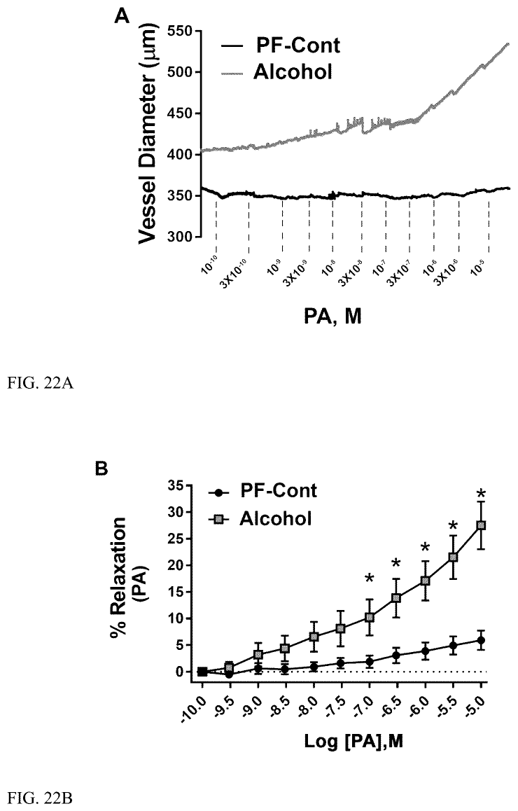

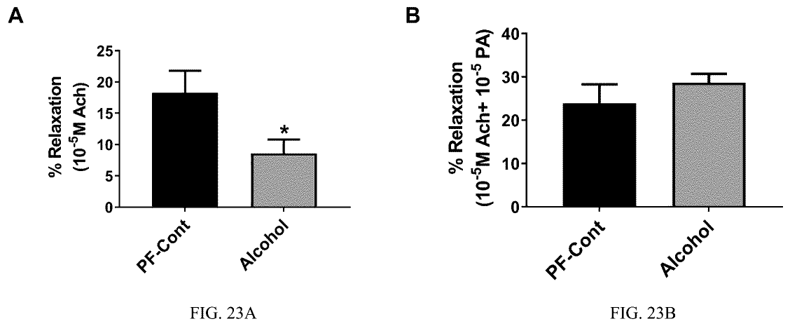

The term “phosphatidic acid” as used herein refers to the molecule labeled “phosphatidic acid” shown in FIG. 9 where each R1 and R2 is independently selected from C16 to C22, e.g. 16:0, 16:1, 16:2, 16:3, 16:4, 18:0, 18:1, 18:2, 18:3, 18:4, 20:0, 20:1, 20:2, 20:3, 20:4, 20:5, 20:6, 22:0, 22:1, 22:2, 22:3, 22:4, 22:5, 22:6, and mixtures of any two or more phosphatidic acid molecules.

Pharmaceutical compositions are provided according to aspects of the present disclosure which include a therapeutically effective amount of a lipid which is downregulated in a pregnant subject exposed to ethanol, and/or a fetus, exposed to ethanol in utero; and a pharmaceutically acceptable excipient. According to aspects of the present disclosure, the lipid included in a pharmaceutical composition is selected from the group consisting of: a glycerophosphethanolamine, a glycerophosphocholine, a triacylglycerol, a phosphosphingolipid, a glycerophosphoinositol, and a glycerophosphoserine.

According to aspects of the present disclosure, the lipid included in a pharmaceutical composition is selected from the group consisting of: a phosphatidic acid, a phosphatidylcholine, a phosphatidylethanolamine, a phosphatidylinositol, and a phosphatidylserine.

Pharmaceutical compositions are provided according to aspects of the present disclosure which include a therapeutically effective amount of a phosphatidic acid which is downregulated in a pregnant subject exposed to ethanol, and/or a fetus, exposed to ethanol in utero; and a pharmaceutically acceptable excipient.

The phrase “exposure to ethanol” relating to a pregnant subject and/or a fetal subject in utero as used herein refers to ethanol intake, typically by oral ingestion, by the pregnant subject, and consequent exposure of the fetus to the ethanol in the maternal system. The terms “ethanol” and “alcohol” as used interchangeably herein refer to ethanol present in typical alcoholic beverages consumed by humans.

A therapeutically effective amount of a lipid which is downregulated in a pregnant subject exposed to ethanol and/or a fetus exposed to ethanol in utero administered according to methods of the present disclosure is an amount which has a beneficial effect in a subject being treated.

A therapeutically effective amount of a phosphatidic acid administered according to methods of the present disclosure, is an amount which has a beneficial effect in a subject being treated.

The dosage of a lipid downregulated in a pregnant subject exposed to ethanol and/or a fetus exposed to ethanol in utero, and in particular embodiments a phosphatidic acid, which is downregulated in a pregnant subject exposed to ethanol and/or a fetus exposed to ethanol in utero, and any optional additional therapeutic agent, will vary based on factors such as, but not limited to, the route of administration; the age, health, and weight of the subject to whom the composition is to be administered; the nature and extent of the subject's symptoms, if any, and the effect desired. Dosage may be adjusted depending on whether treatment is to be acute or continuing. One of skill in the art can determine a pharmaceutically effective amount in view of these and other considerations typical in medical practice.

In general it is contemplated that a daily dosage of a lipid downregulated in a pregnant subject exposed to ethanol and/or a fetus exposed to ethanol in utero, and in particular embodiments a phosphatidic acid, and any optional additional therapeutic agent is in the range of about 0.001 to 100 milligrams per kilogram of a subject's body weight. A daily dose may be administered as two or more divided doses to obtain the desired effect. A pharmaceutical composition including a lipid, and in particular embodiments a phosphatidic acid, and any optional additional therapeutic agent, may also be formulated for sustained release to obtain desired results.

In particular aspects of inventive methods, a lipid downregulated in a pregnant subject exposed to ethanol and/or a fetus exposed to ethanol in utero, and in particular embodiments a phosphatidic acid, is administered in doses of 0.1 mg/day to 1 g/day, such as 0.1 mg/day to 0.25 mg/day, 0.25 mg/day to 0.5 mg/day, 0.5 mg/day to 0.75 mg/day, 0.75 mg/day to 1 mg/day, 0.25 mg/day to 500 mg/day, 0.5 mg/day to 200 mg/day, 0.75 mg/day to 100 mg/day, 1 mg/day to 2 mg/day, such as 2 mg/day to 5 mg/day, 5 mg/day to 10 mg/day, 5 mg/day to 20 mg/day, 10 mg/day to 20 mg/day, 20 mg/day to 30 mg/day, 30 mg/day to 40 mg/day, 40 mg/day to 50 mg/day, 40 mg/day to 60 mg/day, 60 mg/day to 70 mg/day, 70 mg/day to 80 mg/day, 80 mg/day to 90 mg/day, 90 mg/day to 95 mg/day, 95 mg/day to 100 mg/day, 100 mg/day to 150 mg/day, 150 mg/day to 200 mg/day, 200 mg/day to 250 mg/day, 250 mg/day to 300 mg/day, 300 mg/day to 350 mg/day, 350 mg/day to 400 mg/day, 400 mg/day to 450 mg/day, 450 mg/day to 500 mg/day, 500 mg/day to 550 mg/day, 550 mg/day to 600 mg/day, 600 mg/day to 650 mg/day, 650 mg/day to 700 mg/day, 700 mg/day to 750 mg/day, 750 mg/day to 800 mg/day, 800 mg/day to 850 mg/day, 850 mg/day to 900 mg/day, 900 mg/day to 950 mg/day or 950 mg/day to 1 g/day. Such dosages may be increased or decreased depending on factors such as, but not limited to, the route of administration; the age, health, and weight of the subject to whom the composition is to be administered; the nature and extent of the subject's symptoms, if any, and the effect desired. Dosage may be adjusted depending on whether treatment is to be acute or continuing. Dosage may be adjusted depending on how much alcohol the subject has ingested, over what period of time the alcohol was ingested and/or how frequently the alcohol was ingested. One of skill in the art can determine a pharmaceutically effective amount in view of these and other considerations typical in medical practice.

In particular aspects of inventive methods, a phosphatidic acid is administered in doses of 0.1 mg/kg/day to 10 mg/kg/day, such as 0.1 mg/kg/day to 0.2 mg/kg/day, 0.1 mg/kg/day to 0.25 mg/kg/day, 0.1 mg/kg/day to 0.3 mg/kg/day, 0.1 mg/kg/day to 0.4 mg/kg/day, 0.1 mg/kg/day to 0.5 mg/kg/day, 0.1 mg/kg/day to 0.6 mg/kg/day, 0.1 mg/kg/day to 0.7 mg/kg/day, 0.1 mg/kg/day to 0.8 mg/kg/day, such as 0.1 mg/kg/day to 0.9 mg mg/kg/day, 0.1 mg/kg/day to 1 mg/kg/day, 0.5 mg/kg/day to 1 mg/kg/day, 0.5 mg/kg/day to 2 mg/kg/day, 0.5 mg/kg/day to 3 mg/kg/day, 0.5 mg/kg/day to 4 mg/kg/day, 0.5 mg/kg/day to 5 mg/kg/day, 0.5 mg/kg/day to 6 mg/kg/day, 0.5 mg/kg/day to 7 mg/kg/day, 0.5 mg/kg/day to 8 mg/kg/day, 0.5 mg/kg/day to 9 mg/kg/day, 0.5 mg/kg/day to 10 mg/kg/day, 0.5 mg/kg/day to 0.6 mg/kg/day, 0.6 mg/kg/day to 0.7 mg/kg/day, 0.7 mg/kg/day to 0.8 mg/kg/day, 0.8 mg/kg/day to 0.9 mg/kg/day, 0.9 mg/kg/day to 1 mg/kg/day, 1 mg/kg/day to 1.1 mg/kg/day, 0.6 mg/kg/day to 1 mg/kg/day, or 0.7 mg/kg/day to 1 mg/kg/day. Such dosages may be increased or decreased depending on factors such as, but not limited to, the route of administration; the age, health, and weight of the subject to whom the composition is to be administered; the nature and extent of the subject's symptoms, if any, and the effect desired. Dosage may be adjusted depending on whether treatment is to be acute or continuing. Dosage may be adjusted depending on how much alcohol the subject has ingested, over what period of time the alcohol was ingested and/or how frequently the alcohol was ingested. One of skill in the art can determine a pharmaceutically effective amount in view of these and other considerations typical in medical practice.

In particular aspects of inventive methods, a phosphatidic acid is administered in doses of 0.7 mg/kg/day to 1 mg/kg/day.

Dosages for newborns can be determined by application of Clark's rule to specific dosages disclosed herein. Clark's rule is a known pediatric medication dosing rule described in the medical literature that utilizes the patient's weight to calculate medication dosage, see B. J. Delgado et al., Clark's Rule, StatPearls [Internet], 2023, Bookshelf ID: NBK541104PMID: 31082148, StatPearls Publishing LLC.

Additional equations that utilize pediatric weight can be used to calculate medication dosing include Salisbury's rule, Penna's rule, and the Body Surface Area rule.

Clark's rule is an equation used to calculate pediatric medication dosage based on the known weight of a patient and a known adult dose of medication to be used. Clark's rule equation is defined as the weight of the patient in pounds divided by the average standard weight of 150 pounds (68 kg) multiplied by the adult dose of a drug to obtain the pediatric medication dose, as is demonstrated below:

$$( Weight * divided by 150 lbs . ) \times Adult Dose ** = Pediatric Dosage$$

$$( Weight ** * divided by 68 kg ) \times Adult Dose ** = Pediatric Dosage$$

*Weight of pediatric patient in pounds (lbs.)
**Adult dose is the recommended dosage for adult medication use
***Weight of pediatric patient in kilograms (kg)

Methods of the present disclosure include administration of a pharmaceutical composition of the present disclosure by a route of administration including, but not limited to, oral, rectal, nasal, pulmonary, epidural, ocular, otic, intraarterial, intracardiac, intracerebroventricular, intradermal, intravenous, intramuscular, intraperitoneal, intraosseous, intrathecal, intravesical, subcutaneous, topical, transdermal, and transmucosal, such as by sublingual, buccal, vaginal, and inhalational, routes of administration.

In particular aspects of inventive methods, a phosphatidic acid is administered orally in doses of 0.7 mg/kg/day to 1 mg/kg/day.

### Assays of Effectiveness

According to aspects of the present disclosure, one or more correlative biomarkers of therapeutic activity of a lipid downregulated in a pregnant subject exposed to ethanol and/or a fetus exposed to ethanol in utero, and in particular embodiments a phosphatidic acid, in a subject in need thereof are assayed to assess treatment in the subject. Biomarkers include, but are not limited to, assessment of growth of a fetus, assessment of levels of the lipid, and in particular embodiments a phosphatidic acid, in the pregnant subject and/or fetus. Such biomarkers are measured according to standard methodologies, for example as described herein.

According to aspects of the present disclosure, assays for effects of treatment are used to monitor a subject. Thus, for example, a test sample is obtained from: the pregnant subject exposed to ethanol, and/or the fetus exposed to ethanol in utero, and/or the post-natal subject exposed to ethanol in utero, before treatment according to a method of the present disclosure and at one or more times during and/or following treatment in order to assess effectiveness of the treatment. In a further example, a test sample is obtained from the subject or subjects at various times in order to assess the course or progress of disease or healing. Assays may include use of one or more standards and/or controls.

### Biomarkers

Standards and controls suitable for assays are well-known in the art and the standard and/or control used can be any appropriate standard and/or control.

A test sample to be assayed for a biomarker can be any biological fluid, cell or tissue of a subject that includes or is suspected of including the biomarker, illustratively including blood, plasma, serum, urine, saliva, ascites, cerebrospinal fluid, cerebroventricular fluid, pleural fluids, pulmonary and bronchial lavage samples, mucous, sweat, tears, semen, bladder wash samples, amniotic fluid, lymph, peritoneal fluid, synovial fluid, bone marrow aspirate, tumor cells or tissue, organ cells or tissue, such as biopsy material.

A biomarker can be assayed by any of various methodologies including, but not limited to, lipid assay, protein or peptide assay, and nucleic acid assay.

Lipid assays include, but are not limited to, chromatographic methods and mass spectrometric assays. Mass spectrometry devices and general methods of their use are well known in the art as exemplified in McMaster, M., LC/MS A Practical User's Guide, 2005, John Wiley & Sons, USA; and Hoffmann and Stroobant, Mass Spectrometry Principles and Applications, 2007, John Wiley & Sons, England.

Assays for detecting nucleic acids, include, but are not limited to, sequencing; polymerase chain reactions (PCR) such as RT-PCR; dot blot; in situ hybridization; Northern blot; and RNase protection.

Immunoassay methods include, but are not limited to, enzyme-linked immunosorbent assay (ELISA), enzyme-linked immunofiltration assay (ELIFA), flow cytometry, immunoblot, immunoprecipitation, immunohistochemistry, immunocytochemistry, luminescent immunoassay (LIA), fluorescent immunoassay (FIA), and radioimmunoassay.

### Additional Therapeutic Agent

One or more additional therapeutic agents is administered according to aspects of the present disclosure.

The term “additional therapeutic agent” is used herein to refer to a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues which is a biologically, physiologically, or pharmacologically active substance (or substances) that acts locally or systemically in a subject.

Additional therapeutic agents included according to aspects of methods and compositions of the present disclosure include, but are not limited to, antibiotics, antivirals, antineoplastic agents, analgesics, antipyretics, antidepressants, antipsychotics, anti-cancer agents, antihistamines, anti-osteoporosis agents, anti-osteonecrosis agents, antiinflammatory agents, anxiolytics, chemotherapeutic agents, diuretics, growth factors, hormones, non-steroidal antiinflammatory agents, steroids and vasoactive agents.

### Subject

A subject treated according to methods and using compositions of the present disclosure are typically mammals. A mammalian subject can be any mammal including, but not limited to, a human; a non-human primate; a rodent such as a mouse, rat, or guinea pig; a domesticated pet such as a cat or dog; a horse, cow, pig, sheep, goat, or rabbit. In aspects of the present disclosure, the subject is human. The terms “subject” and “patient” are used interchangeably herein.

### Compositions/Formulations

Compositions are provided according to aspects of the present disclosure which include: a therapeutically effective amount of a lipid which is downregulated in a pregnant subject exposed to ethanol, and/or a fetus, exposed to ethanol in utero; and a pharmaceutically acceptable excipient.

A pharmaceutical composition of the present disclosure may be in any dosage form suitable for administration to a subject, illustratively including solid, semi-solid and liquid dosage forms such as tablets, capsules, powders, granules, suppositories, pills, solutions, suspensions, ointments, lotions, creams, gels, pastes, sprays and aerosols. Liposomes and emulsions can be used to deliver a pharmaceutical agent, particularly a hydrophobic pharmaceutical agent.

Pharmaceutical compositions of the present disclosure include a pharmaceutically acceptable carrier such as an excipient, diluent and/or vehicle.

Delayed release formulations of compositions and delayed release systems, such as semipermeable matrices of solid hydrophobic polymers can be used.

The term “pharmaceutically acceptable carrier” refers to a carrier which is suitable for use in a subject without undue toxicity or irritation to the subject and which is compatible with other ingredients included in a pharmaceutical composition.

Pharmaceutically acceptable carriers, methods for making pharmaceutical compositions and various dosage forms, as well as modes of administration are well-known in the art, for example as detailed in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, PA: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

A solid dosage form for administration or for suspension in a liquid prior to administration illustratively includes capsules, tablets, powders, and granules. In such solid dosage forms, one or more active agents, is admixed with at least one carrier illustratively including a buffer such as, for example, sodium citrate or an alkali metal phosphate illustratively including sodium phosphates, potassium phosphates and calcium phosphates; a filler such as, for example, starch, lactose, sucrose, glucose, mannitol, and silicic acid; a binder such as, for example, carboxymethylcellulose, alginates, gelatin, polyvinylpyrrolidone, sucrose, and acacia; a humectant such as, for example, glycerol; a disintegrating agent such as, for example, agar-agar, calcium carbonate, plant starches such as potato or tapioca starch, alginic acid, certain complex silicates, and sodium carbonate; a solution retarder such as, for example, paraffin; an absorption accelerator such as, for example, a quaternary ammonium compound; a wetting agent such as, for example, cetyl alcohol, glycerol monostearate, and a glycol; an adsorbent such as, for example, kaolin and bentonite; a lubricant such as, for example, talc, calcium stearate, magnesium stearate, a solid polyethylene glycol or sodium lauryl sulfate; a preservative such as an antibacterial agent and an antifungal agent, including for example, sorbic acid, gentamycin and phenol; and a stabilizer such as, for example, sucrose, EDTA, EGTA, and an antioxidant.

Solid dosage forms optionally include a coating such as an enteric coating. The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied having a thickness such that the entire coating does not dissolve in the gastrointestinal fluids at pH below 3 associated with stomach acids, yet dissolves above pH 3 in the small intestine environment. It is expected that any anionic polymer exhibiting a pH-dependent solubility profile is readily used as an enteric coating in the practice of the present disclosure to achieve delivery of the active agent to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and case of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof. A particular enteric coating material includes acrylic acid polymers and copolymers described for example U.S. Pat. No. 6,136,345.

The enteric coating optionally contains a plasticizer to prevent the formation of pores and cracks that allow the penetration of the gastric fluids into the solid dosage form. Suitable plasticizers illustratively include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g. hydroxypropylcellulose, acids or bases) to solubilize or disperse the coating material, and to improve coating performance and the coated product.

Liquid dosage forms for oral administration include one or more active agents and a pharmaceutically acceptable carrier formulated as an emulsion, solution, suspension, syrup, or elixir. A liquid dosage form of a composition of the present disclosure may include a colorant, a stabilizer, a wetting agent, an emulsifying agent, a suspending agent, a sweetener, a flavoring, or a perfuming agent.

For example, a composition for parenteral administration may be formulated as an injectable liquid. Examples of suitable aqueous and nonaqueous carriers include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of a desirable particle size in the case of dispersions, and/or by the use of a surfactant, such as sodium lauryl sulfate. A stabilizer is optionally included such as, for example, sucrose, EDTA, EGTA, and an antioxidant.

According to aspects of the present disclosure, a composition for parenteral administration includes an aqueous carrier and a phosphatidic acid.

According to aspects of the present disclosure, a composition for parenteral administration includes an aqueous carrier, 0.1%-10% serum albumin, and a phosphatidic acid. An included serum albumin can be derived from serum of any species compatible with the subject to whom the composition is to be administered, such as bovine serum albumin, human serum albumin, and the like.

For topical administration, a composition can be formulated for administration to the skin such as for local effect, and/or as a “patch” formulation for transdermal delivery. Pharmaceutical formulations suitable for topical administration include, for example, ointments, lotions, creams, gels, pastes, sprays and powders. Ointments, lotions, creams, gels and pastes can include, in addition to one or more active agents, a base such as an absorption base, water-removable base, water-soluble base or oleaginous base and excipients such as a thickening agent, a gelling agent, a colorant, a stabilizer, an emulsifying agent, a suspending agent, a sweetener, a flavoring, or a perfuming agent.

Transdermal formulations can include percutaneous absorption enhancers such as acetone, azone, dimethyl acetamide, dimethyl formamide, dimethyl sulfoxide, ethanol, oleic acid, polyethylene glycol, propylene glycol and sodium lauryl sulfate. Ionotophoresis and/or sonophoresis can be used to enhance transdermal delivery.

Powders and sprays for topical administration of one or more active agents can include excipients such as talc, lactose and one or more silicic acids. Sprays can include a pharmaceutical propellant such as a fluorinated hydrocarbon propellant, carbon dioxide, or a suitable gas. Alternatively, a spray can be delivered from a pump-style spray device which does not require a propellant. A spray device delivers a metered dose of a composition contained therein, for example, using a valve for regulation of a delivered amount.

Suitable surface-active agents useful as a pharmaceutically acceptable carrier or excipient in the pharmaceutical compositions of the present disclosure include non-ionic, cationic and/or anionic surfactants having good emulsifying, dispersing and/or wetting properties. Suitable anionic surfactants include both water-soluble soaps and water-soluble synthetic surface-active agents. Suitable soaps are alkaline or alkaline-earth metal salts, non-substituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, non-substituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphthalene sulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidylcholine, dipalmitoylphosphatidyl-choline and their mixtures.

Suitable non-ionic surfactants useful as pharmaceutically acceptable carriers or excipients in the pharmaceutical compositions of the present disclosure include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolycthoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants useful as pharmaceutically acceptable carriers or excipients in the pharmaceutical compositions of the present disclosure include quaternary ammonium salts, preferably halides, having 4 hydrocarbon radicals optionally substituted with halo, phenyl, substituted phenyl or hydroxy; for instance quaternary ammonium salts containing as N-substituent at least one C8-C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, New Jersey, 1981), “Tensid-Taschenbuch”, 2nd ed. (Hanser Verlag, Vienna, 1981) and “Encyclopaedia of Surfactants (Chemical Publishing Co., New York, 1981).

Structure-forming, thickening or gel-forming agents may be included into the pharmaceutical compositions and combined preparations of the disclosure. Suitable such agents are in particular highly dispersed silicic acid, such as the product commercially available under the trade name Acrosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

In particular aspects, a pharmaceutically acceptable carrier is a particulate carrier such as lipid particles including liposomes, micelles, unilamellar or mulitlamellar vesicles; polymer particles such as hydrogel particles, polyglycolic acid particles or polylactic acid particles; inorganic particles such as calcium phosphate particles such as described in for example U.S. U.S. Pat. No. 5,648,097; and inorganic/organic particulate carriers such as described for example in U.S. Pat. No. 6,630,486.

A particulate pharmaceutically acceptable carrier can be selected from among a lipid particle; a polymer particle; an inorganic particle; and an inorganic/organic particle. A mixture of particle types can also be included as a particulate pharmaceutically acceptable carrier.

A particulate carrier is typically formulated such that particles have an average particle size in the range of about 1 nm-10 microns. In particular aspects, a particulate carrier is formulated such that particles have an average particle size in the range of about 1 nm-100 nm.

Detailed information concerning customary ingredients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, PA: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

According to aspects of the present disclosure, a lipid administered to a subject and/or included in a pharmaceutical composition is purified. The term “purified” as used herein refers to a material which has been treated to remove one or more other components with which the lipid is associated in its natural environment, during synthetic processes, and the like. A purified lipid is at least 75% free, at least 80% free, at least 85% free, at least 90% free, at least 95% free of other components. A purified lipid constitutes at least about 50%, 60%, 70%, 80%, 90%, 95%, 96%, 97%, 98%, 99%, or greater % of a lipid composition, e.g. prior to adding a pharmaceutically acceptable carrier.

Embodiments of inventive compositions and methods are illustrated in the following examples. These examples are provided for illustrative purposes and are not considered limitations on the scope of inventive compositions and methods.

### EXAMPLES

### Example 1

In this example, ethanol was administered to pregnant rats: once-daily gavage, GD 4-10:4.5 g/kg, BAC, 216 mg/dl; GD 11-20:6 g/kg, BAC, 289 mg/dl.

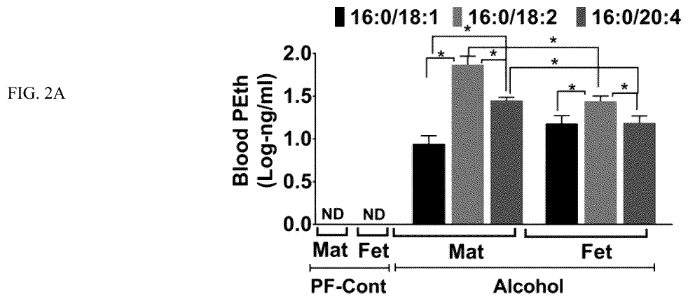

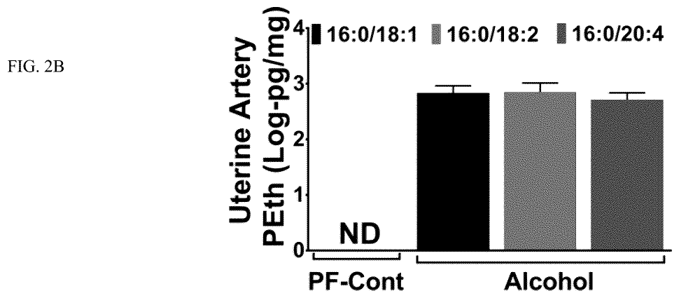

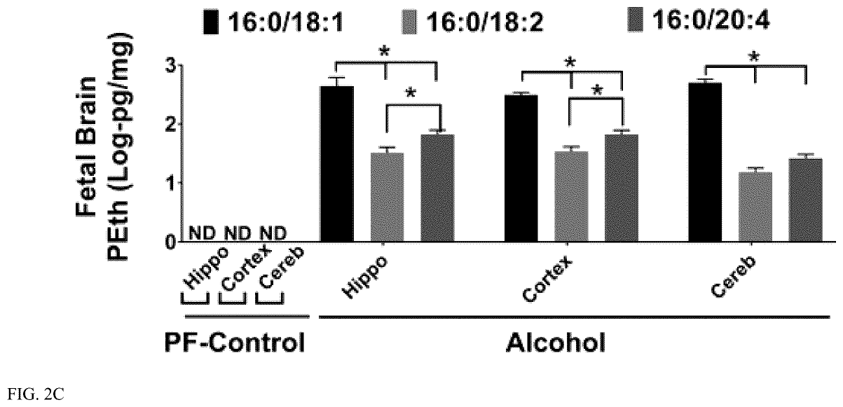

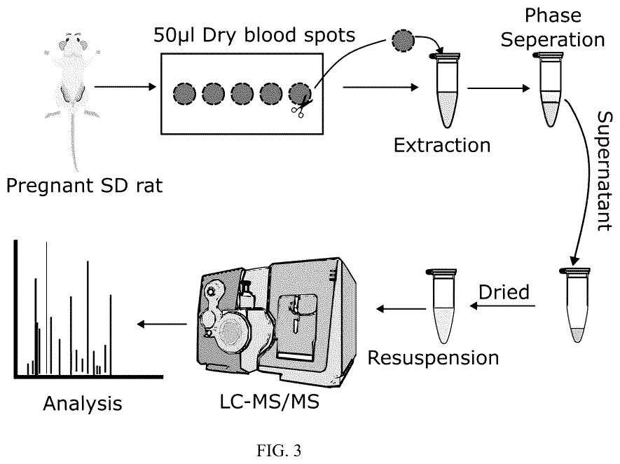

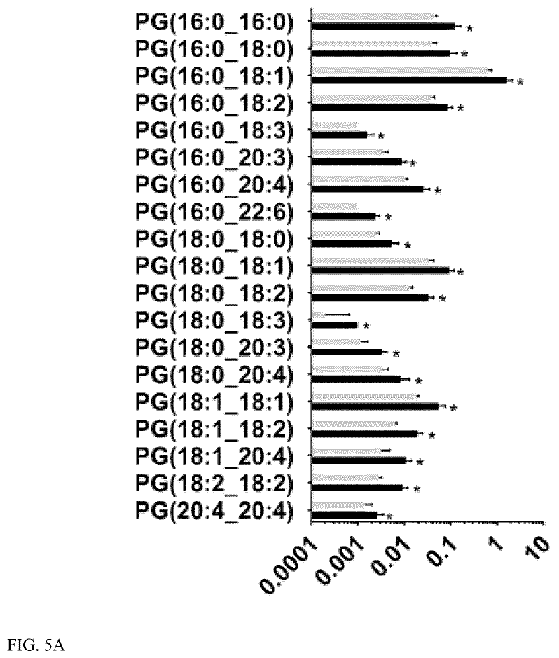

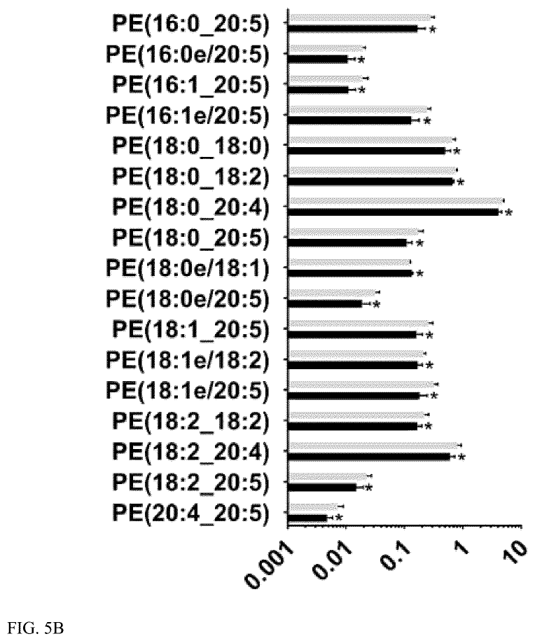

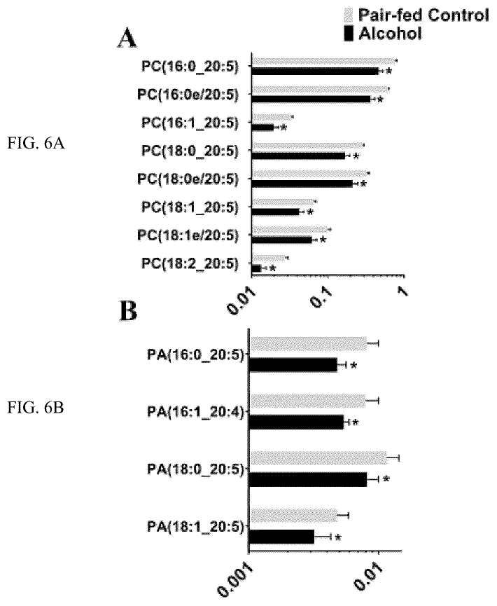

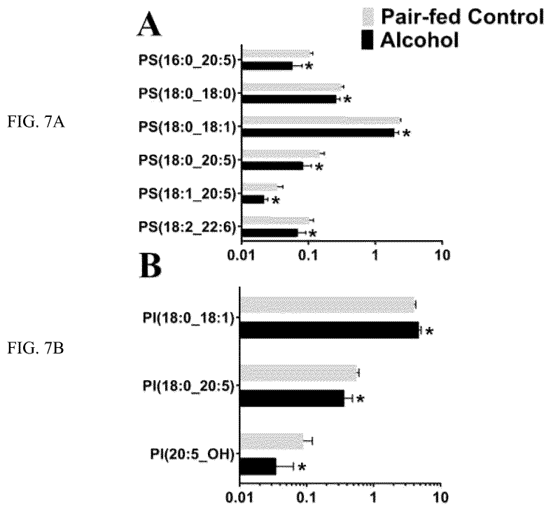

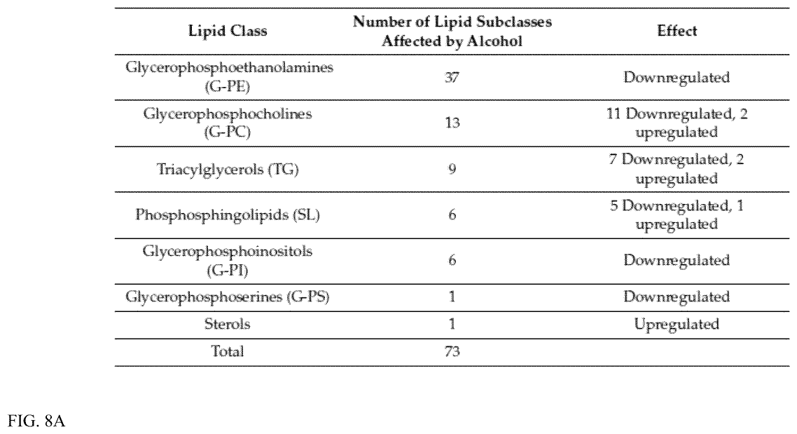

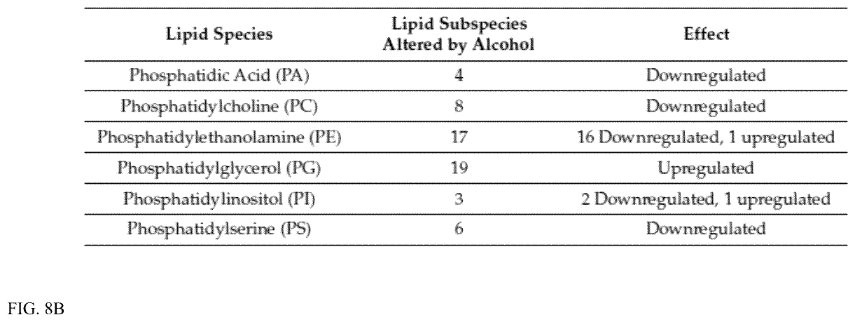

It was found that ethanol exposure affects lipid content of both maternal and fetal samples, see FIGS. 2A, 2B, 2C, 4A, 4B, 4C, 4D, 5A, 5B, 6, 7, 8A, and 8B.

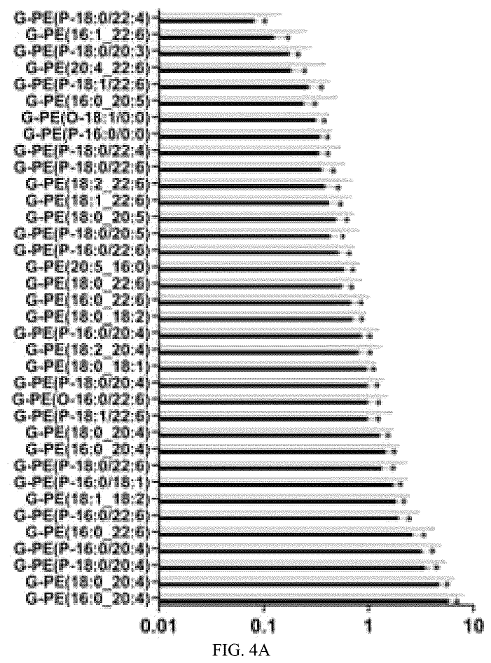

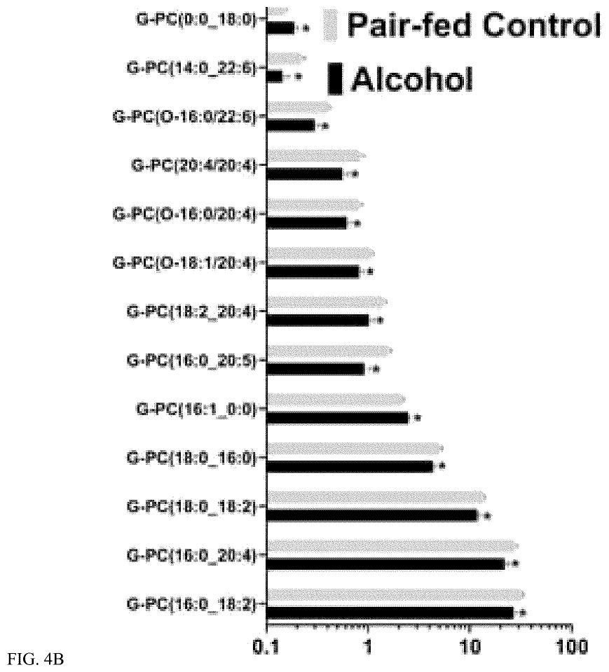

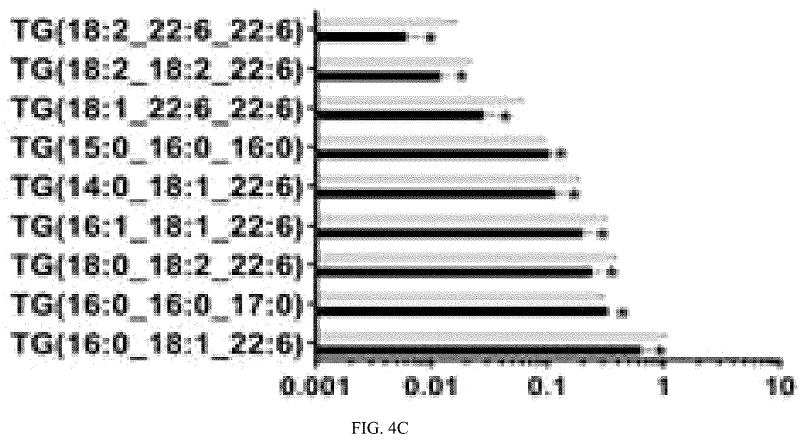

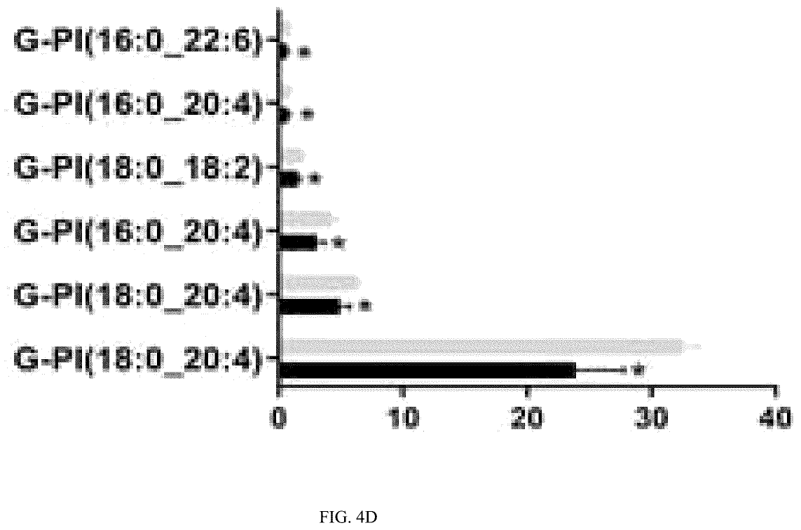

Untargeted lipidomics showed 73 of 326 lipids were altered in ethanol-exposed subjects and targeted lipidomics showed alterations in PA and related phosphatidylcholine, phosphatidylethanolamine, phosphatidylglycerol. FIGS. 4A-4D show that 73 lipids were affected by ethanol of which 67 were downregulated and 6 were upregulated. The four most affected lipid species were Glycerophosphoethanolamines (G-PE;A), Glycerophosphocholines (G-PC;B), Triacylglycerols (TG;C), and Glycerophosphoinositols (G-PI;D). All Phosphatidylglycerol subspecies that were affected were upregulated. Phosphatidylethanolamine (PE), 16 of the 17 affected were downregulated, All affected species of Phosphatidylcholine & Phosphatidic Acid downregulated. Six subspecies of Phosphatidylserine were downregulated. Two of three subspecies of Phosphatidylinositol (PI) downregulated.

Ethanol is transphosphatidylized to phosphatidylethanol (PEth) at the expense of phosphatidic acid (PA), see FIG. 9.

Mean fetal weight, and crown-rump length of the fetuses from ethanol-fed rats were ˜9% and ˜8% lower (P<0.05) than the pair-fed control animal, respectively. Administration of phosphatidic acid to the pregnant animals, 4.5 mg/kg, GD 5-10; 6 mg/kg GD 11-20, concomitant with ethanol completely rescued the FASD growth deficits.

Acetylcholine-induced uterine artery relaxation was significantly impaired in ethanol-administered rats (P<0.05), and in vivo PA administration completely reversed the ethanol-induced vascular dysfunction. Molecule administered: PA (#840857C, Avanti Polar Lipids) was air-dried under a gentle stream of nitrogen gas and resuspended in 1% BSA to make a 10⁻² M stock solution. PA was administered to pregnant rats in vivo at a dose of 4.5 mg/kg/day for alcohol dose of 4.5 g/kg body weight from gestation day 4 through 10 and 6 mg/kg/day for alcohol dose of 6 g/kg body weight from gestation day 11 through 19 or 20.

To validate specificity and direct actions of PA, ex vivo supplementation of 10⁻⁵ M PA was found to similarly reverse ethanol-induced vasodilatory deficit in animals; no difference was detected after PA treatment between pair-fed control and ethanol groups (P=0.37) with significant interaction between PA concentrations and ethanol exposure (PA X Alcohol, P<0.0001).

Ethanol significantly reduced vasodilatory P-Ser1177 endothelial nitric oxide synthase (cNOS) levels in the uterine artery (P<0.05). PA treatment significantly reversed P-Ser1177 eNOS level in ethanol-exposed uterine arteries (P<0.05) following ex vivo PA or in vivo PA. Neither ethanol treatment nor PA affected total eNOS levels.

For ex vivo examples, 16:0-18:1 PA (1-palmitoyl-2-oleoyl-sn-glycero-3-phosphate (sodium salt; CAS Number 169437-35-8) was used and administered to the ex vivo tissues. For in vivo examples, L-α-phosphatidic acid derived from chicken eggs was used, sodium salt; CAS Number 383907-53-7, which is a natural lipid mixture with fatty acid distribution: 16:0—34.2%; 16:1—1%, 18:0—11.5% 18:1—31.5%; 18:2—18.5%; 20:4—2.7%, and 22:6—0.7%, average molecular weight 706.158.

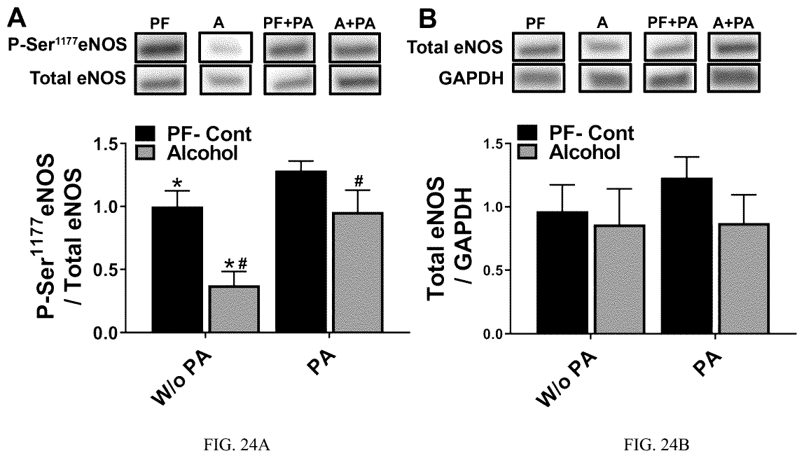

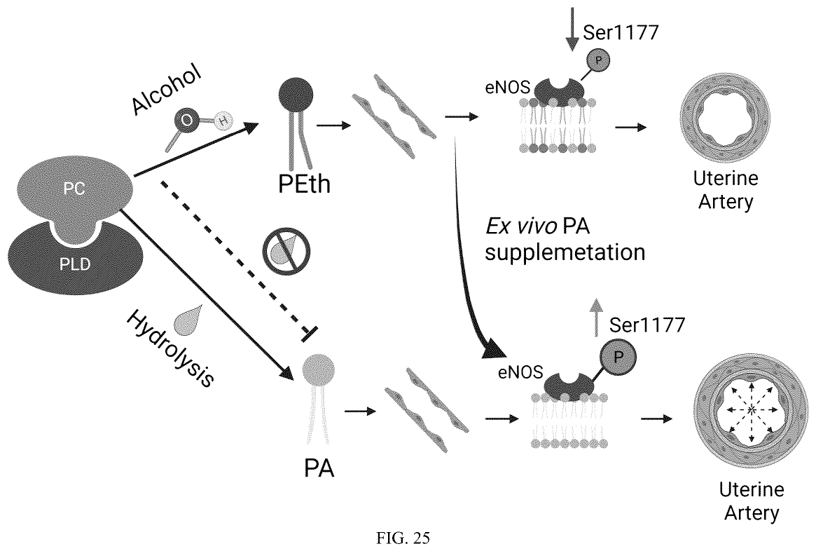

Thus, PA, a direct target of alcohol metabolism, ameliorates ethanol-induced vascular dysfunction of the maternal uterine artery and fetal growth deficits see FIGS. 24A, 24B, 25, 26A-E, 27A-D, and 28-31.

These data provide the first evidence of the interaction of the simplest lipid molecule (PA) with ethanol on FASD growth phenotype and maternal uterine artery vascular function. Overall, these results demonstrate that PA ameliorates FASD intrauterine growth restriction pathogenesis.

### Example Summary

Untargeted and targeted lipidomics: a two-pronged approach to identify gestational chronic binge ethanol effects on blood lipid levels in a FASD rat model. In targeted analysis, Phosphatidylcholine (PC), Phosphatidylethanolamine (PE), Phosphatidylglycerol (PG), Phosphatidic Acid (PA), Phosphatidylinositol (PI), and Phosphatidylserine (PS); 36 of these were downregulated and 21 lipid subspecies were upregulated.

Uterine artery proteome: Novel complimentary quantitative mass spectrometric approaches including non-labeled (label-free) nano LC MS/MS, gel-based 2-D DIGE MALDI TOF/TOF, and label-based methods like iTRAQ nano LC MS/MS to study differential protein signatures during pregnancy and validated to show ethanol inhibits vasodilatory pathways.

PEth Mass spectrometry: All major PEth homologues were increased in maternal and fetal blood following chronic gestational binge ethanol exposure; homologue distribution profiles were tissue-specific. These were accompanied by concomitant decreases in phosphatidic acid in the maternal blood.

Functional adaptations and lipid effects: phosphatidic acid, a direct molecular target of ethanol metabolism, was used to study PA's effect on uterine vascular function. These results demonstrate that ex vivo PA ameliorates ethanol-induced uterine artery dysfunction. Further, PA reverses ethanol-induced deficits in uterine artery eNOS activity index; In vivo PA ameliorates FASD growth deficits and uterine artery function.

### Example 2

### Materials and Methods

### Treatment Groups and Alcohol/PA In Vivo Dosing Paradigm

Timed pregnant Sprague-Dawley rats (8-12 weeks old), were purchased from Charles River (Wilmington, MA, USA) and arrived on GD 4, where they were housed in a temperature-controlled room (23° C.) with a 12:12 h light-dark cycle. The dams were acclimatized for a day before weighing and handling. The dams were then assigned into experimental groups. Four in vivo treatment groups were utilized: (1) a nutritional pair-fed control group (control), that served as a control for nutrition and for the gavage procedure. To control for the calories derived from alcohol, these pair-fed control rats were administered isocaloric maltose-dextrin (once-daily) via orogastric gavage. (2) A binge alcohol group (alcohol), where dams were acclimatized with a once-daily gavage (orogastric) of 4.5 g/kg ethanol (22.5% weight/volume; peak blood alcohol concentration (BAC), 216 mg/dL) from gestational days (GD) 5-10, and progressed to a 6 g/kg alcohol from GD 11 to 19 (28.5% weight/volume; peak BAC, 289 mg/dL). (3) An in vivo phosphatidic acid (PA) control group (control PA), to control for the in vivo PA supplement. These dams were similar to the nutritional pair-fed control group except that they were administered PA via an intragastric gavage along with the maltose-dextrin. (4) A binge alcohol in vivo PA group (alcohol PA), that received alcohol via intragastric gavage similar to those in the binge alcohol group along with the PA supplement. Daily PA doses were calculated based on the dam's weight at the time of administration (PA (μL)=0.2×dam weight (g) GD 5-10; PA (μL)=0.2105×weight (g) GD 11-19). Intake of food in the alcohol treatment group was measured, and an equivalent amount of food was given to the pair-fed control dams to account for additional nutritional factors. Rats were euthanized by decapitation while under isoflurane anesthesia.

### Maternal and Fetal Weight Measurements

Maternal weights and fetal weights were measured on GD 20-21, one day after the last treatment on GD 19-20, following euthanasia.

### Reagent Preparation

HEPES-Bicarbonate Solution (HBS) (NaCl 130 mM; MgSO_{4.7}H₂O 2.5 mM; KCl 4 mM; CaCl₂) 2.4 mM; NaHCO₃ 4.05 mM; KH₂PO₄ 1.18 mM; HEPES 10 mM; EDTA 0.024 mM; Glucose 6 mM; pH 7.4) was prepared fresh on the day of experiment. PA (#840857C, Avanti Polar Lipids) was suspended in 1% Bovine Serum Albumin (BSA) to make a 10⁻² M stock solution, then prepared in two separate 2.25% and 2.85% solutions, aliquoted for dosage. Acetylcholine (ACh) and Thromboxane (Tbx) stocks were prepared using standard procedures in HBS.

### Arteriography

Following euthanasia, the entirety of the uterus was excised and immediately placed in ice-cold HBS for pressure arteriography experiments. Uterine artery functional assessments were performed following uterine artery isolation using established procedures. Briefly, the uterine horn was transferred to a 200 mm petri dish with solidified Sylgard to facilitate tissue isolation and cleaning in ice-cold HBS. A 3-5 mm segment of the primary uterine artery was dissected between arterial bifurcations from the approximate center of the uterine horn. Surrounding adipose and connective tissues were carefully removed from the uterine artery segment. Through the dual-chamber setup, arterial segments were mounted simultaneously from a treatment (alcohol or alcohol PA) and the respective control (control or control PA) groups, ensuring identical treatment per experiment. The dual-chamber, with mounted vessels, was put in a closed enclosure with 37° C. ambient temperature, and the cannulation setup was completed to allow continuous circulation of a pre-warmed HBS bath. Intramural pressure was increased to 60 mm Hg until the vessels exhibited a myogenic tone (˜15-20 min). Following equilibration, the circulation buffer was changed to fresh HBS warmed to 37° C. Intramural pressure was then increased to mimic in vivo pressures at 90 mm Hg, at which pressure the ACh concentration responses were measured. Vessels were pre-constricted with 10⁻⁶ M Tbx as determined previously. Vessels that failed to demonstrate myogenic tone or did not respond to Tbx were discarded. The above treatment was followed by the administration of three-fold increasing concentrations of ACh from 10⁻¹⁰ M up to 10⁻⁵ M. Vascular response was recorded by established methods using Ionwizard software version 6.6 (Ionoptix LLC, Westwood, MA, USA) for at least 5 min, or until arterial diameter stabilized.

### Immunoblotting

Immunoblotting was performed using standard laboratory procedures. Following euthanasia, the uterine arteries were isolated by separating the vein, and cleaning the adipose tissues in HBS, before flash freezing for immunoblot analysis. Tissues were first homogenized using a 4° C. cooled bead homogenizer (Benchmark Scientific, Sayreville, NJ, USA) and then quantified using BCA protein quantification assay. Next, 20 μg of the uterine artery sample protein was then loaded on to 4-20% mini-protean TGX gels (Bio-rad, Hercules, CA, USA). Following transfer to a PVDF membrane, P-Ser1177 eNOS (Novus Biologicals, Centennial, CO, USA), total eNOS (BD Biosciences, Franklin Lakes, NJ, USA), and β-Actin (Sigma Aldrich, St. Louis, MI, USA) were probed. Densitometry analysis was performed using AzureSpot (Azure Biosystems, Dublin, CA, USA).

### Immunofluorescence

Immunofluorescence assessments were performed using previously published protocols. In brief, maternal uterine arteries were sectioned at 8 μm with a Leica cryostat (CM1860, Leica Biosystems, Buffalo Grove, IL, USA). Sections were subsequently fixed with ice-cold methanol (30 min, −20° C.), and then rinsed in PBS, and incubated in 10% normal serum (60 min). This was followed by incubation with (1:100; p1177-eNOS; Cell Signaling and 1:250; total eNOS, BD Biosciences) primary antibody overnight at 4° C. in a humidified chamber. The sections were then incubated with goat anti-rabbit IgG secondary antibody (Alexa Fluor 488, Invitrogen, Carlsbad, CA, USA), for 1 h at room temperature. Digital images were captured with an Olympus BX63 stereomicroscope that was equipped with U-HGLGPS fluorescent light source, ORCA-Flash 4.0 LT camera, Hamamatsu Photonics (Hamamatsu, Japan), and Olympus cellSens Dimension software Version 3.2 (Olympus, Tokyo, Japan).

### Statistics

Maternal weight and fetal weight (unit of analysis is a dam or litter) were analyzed using two-way mixed ANOVA with alcohol as the between factor and PA as the within factor. Normality was tested using the Shapiro-Wilk normality test where appropriate. Uterine vascular response to ACh was analyzed using two-way ANOVA, followed by multiple comparisons using Fisher's LSD. Data for the ACh concentration response following ANOVA were reported as mean±SEM. Non-linear regression curve fit was performed using a three-parameter equation, Y=Baseline+(Max Response-Baseline)/1+10 (^{LogEC50-X}) to obtain the effective concentration (EC50). The data were considered significant if the p value was <0.05.

### Results

### Growth Assessment

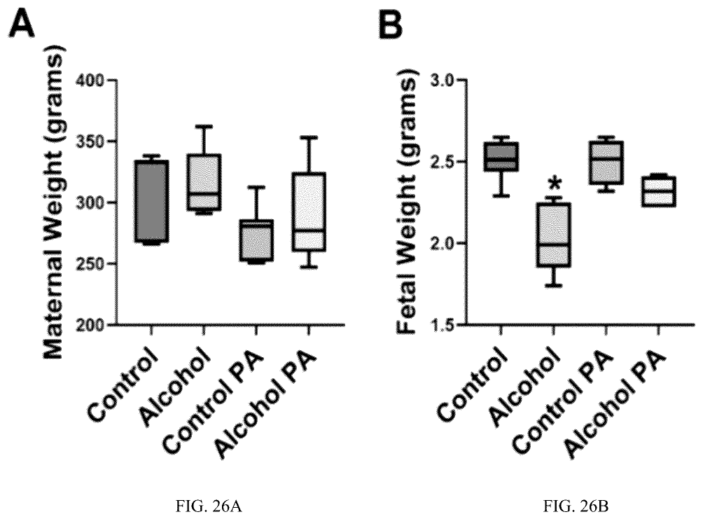

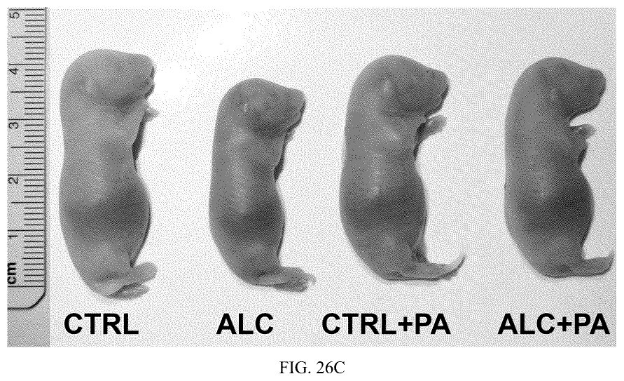

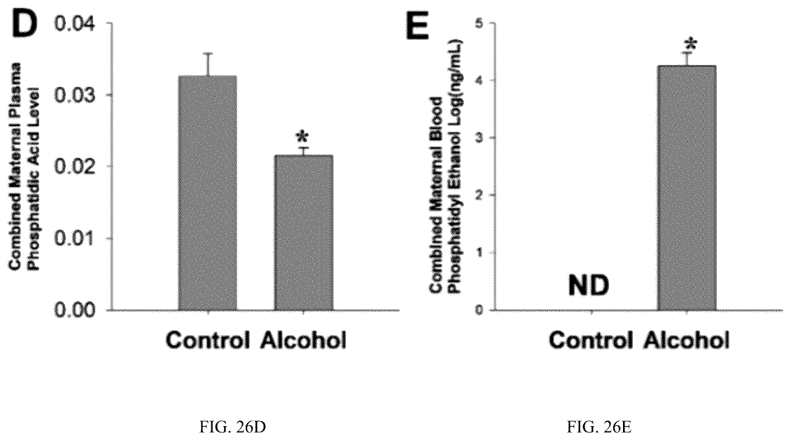

Maternal body weights did not differ among the treatment groups (FIG. 26A). Mean fetal weights were significantly lower in the alcohol group compared with those in the control (p<0.0001), however, the fetal weight difference between the alcohol and the pair-fed control group was completely abolished by concomitant in vivo PA administration with alcohol, indicating reversal of classic FASD growth restriction phenotype (FIG. 26B). The control PA group was not different to the alcohol PA group. Representative fetuses that are in the median weight range in each of the four treatment groups are depicted in FIG. 26C. Litter size among all groups in the cohort were not significantly different (average litter size, control, 10.83±2.56; alcohol, 10.83±1.33; control PA, 10.00±1.54; alcohol PA, 10.00±1.09).

### Phosphatidic Acid and Phosphatidyl Ethanol Assessments

Following mass spectrometry, subspecies of maternal plasma PA and maternal blood PEth in were identified. The combined abundance of PA was evaluated (FIG. 26D) as well as PEth (FIG. 26E) since the combined PEth is a powerful biomarker for alcohol consumption in humans. The total PA level was significantly lower in the alcohol group compared to the controls (p=0.011) and this was accompanied by concomitant increases in levels of total PEth confirming that PEth was formed at the expense of PA as PEth was not detected as expected in the control dams.

### In Vivo PA Supplementation and Reversal of Alcohol-Induced Vascular Dysfunction

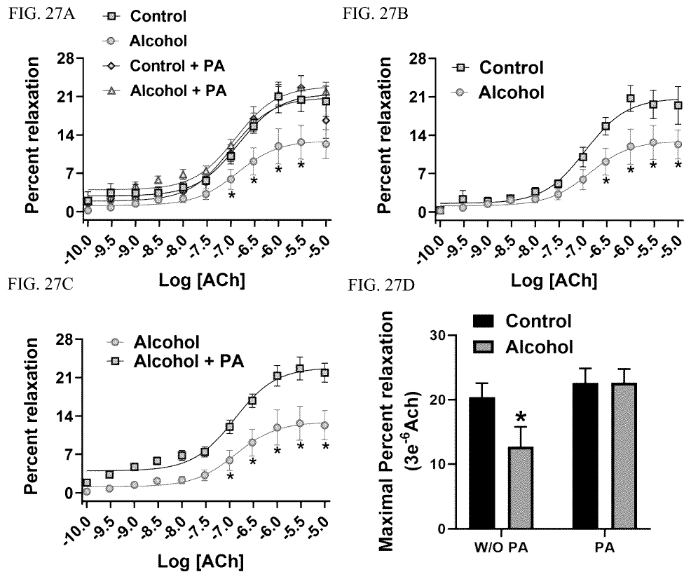

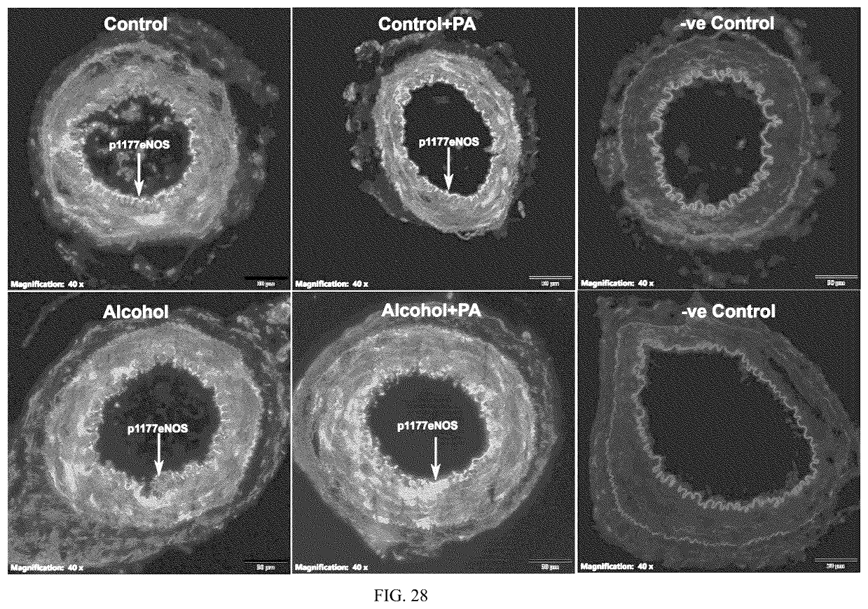

Following in vivo administration of binge alcohol with or without concomitant in vivo PA throughout gestation, vascular function was assessed using pressure arteriography. An interaction of ACh dose X in vivo treatments was noted (FIG. 27A). A significant main effect of ACh dose (p<0.0001) and the in vivo treatment (p<0.0001) were also noted. Vasodilation following ACh, an endothelium-dependent agonist, was significantly decreased in uterine arteries of the alcohol group compared with those in the controls (p<0.05, FIG. 27B). In vivo supplementation of PA throughout pregnancy abolished alcohol-induced decreases in uterine artery vasodilation (FIG. 27C). PA was administered in vivo in the absence of alcohol in the control PA group and no differences in the vasodilation between the control PA and alcohol PA groups were detected (FIG. 27A). Maximal ACh-induced was significantly different in the alcohol group compared to the control, control PA, and alcohol PA groups (p<0.05; FIG. 27D).

Alcohol-Induced Decreases in Stimulatory eNOS Phosphorylation

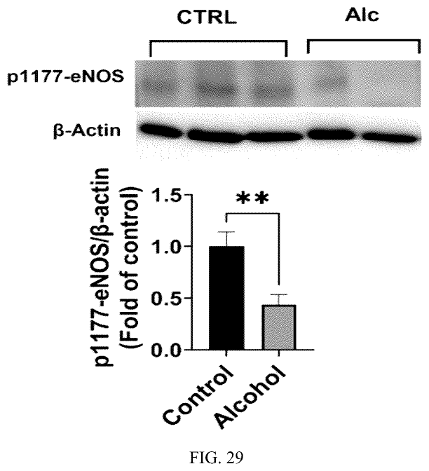

Immunofluorescence imaging demonstrated that excitatory p1177-eNOS was detected in the endothelium of the uterine artery. The level of florescence showed decreases in the levels of phosphorylation at the p1177-eNOS, and was validated by immunoblotting analysis. Alcohol significantly reduced the proportion of phosphorylated p1177-eNOS in the uterine artery (FIG. 29).

In Vivo Phosphatidic Acid (PA) Reversed Alcohol-Induced Decreases in Stimulatory eNOS Phosphorylation

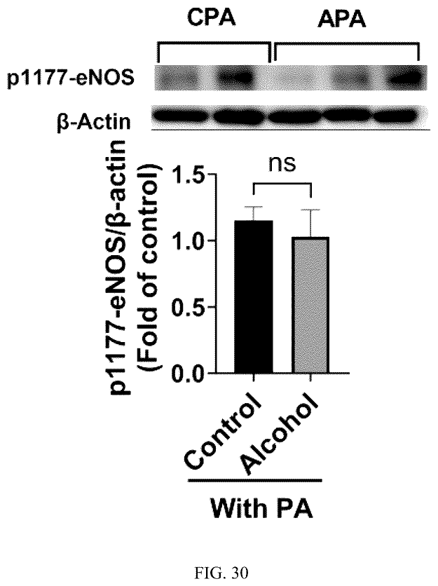

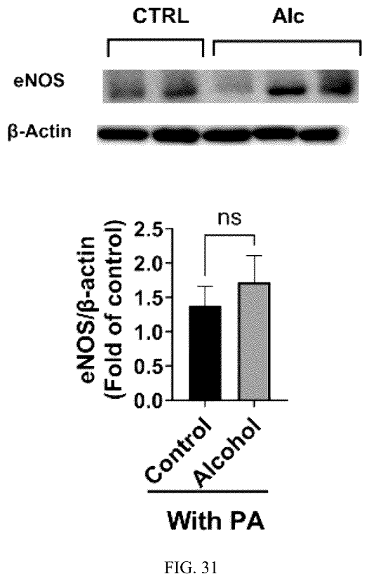

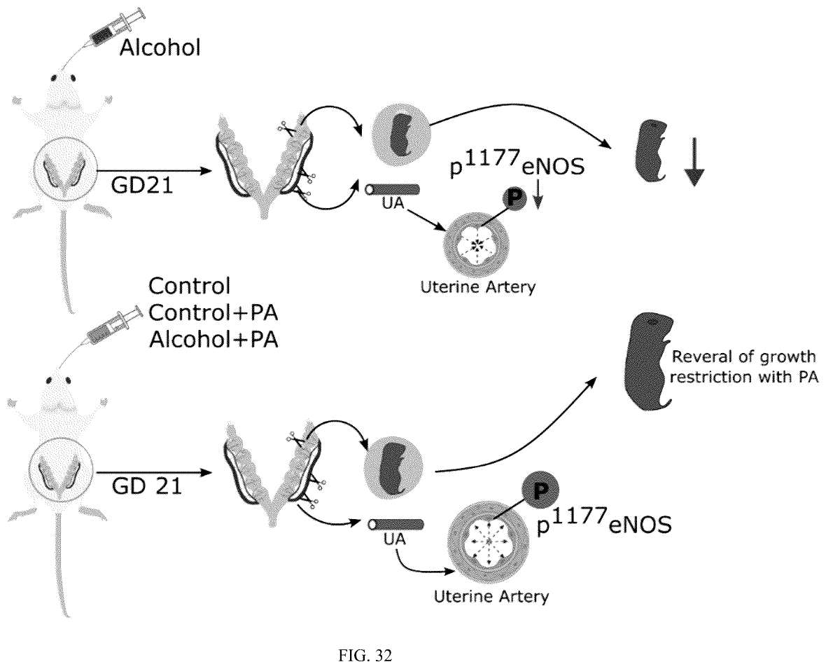

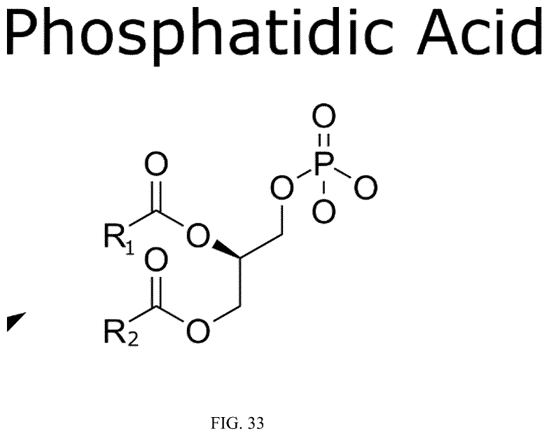

When comparing p1177-eNOS normalized to B actin, the alcohol-induced downregulation of p1177-eNOS was completely reversed following in vivo PA supplementation. p1177-eNOS relative to B actin in the control group was not different between the control PA and the alcohol PA groups (FIG. 30). As shown in FIG. 31, total eNOS was not different among groups. Neither alcohol nor PA had any effect on total eNOS. Further, immunofluorescence showed that total eNOS was also localized to the uterine artery endothelium.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of ameliorating one or more effects of exposure to ethanol in a pregnant subject, and/or a fetal subject in utero, and/or in a post-natal subject, who was exposed to ethanol in utero, comprising:
administering, to the pregnant subject, and/or the fetal subject, and/or post-natal subject, a therapeutically effective amount of a lipid which is downregulated in a pregnant subject exposed to ethanol and/or a fetus exposed to ethanol in utero.

2. The method of claim 1, wherein the lipid is selected from the group consisting of: a glycerophosphethanolamine, a glycerophosphocholine, a triacylglycerol, a phosphosphingolipid, a glycerophosphoinositol, and a glycerophosphoserine.

3. The method of claim 1 wherein the lipid is selected from the group consisting of: a phosphatidic acid, a phosphatidylcholine, a phosphatidylethanolamine, a phosphatidylinositol, and a phosphatidylserine.

4. The method of claim 1 wherein the lipid is a phosphatidic acid.

5. The method of claim 1, wherein the lipid is a phosphatidic acid, or a mixture of phosphatidic acids, each phosphatidic acid having the chemical structure: 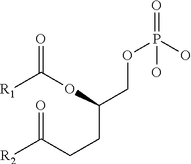
where each R1 and R2 is independently selected from saturated or unsaturated C16 to C22 phosphatidic acid.

6. A pharmaceutical composition, comprising: a therapeutically effective amount of a lipid which is downregulated in a pregnant subject exposed to ethanol, and/or a fetus, exposed to ethanol in utero; and a pharmaceutically acceptable excipient.

7. The pharmaceutical composition of claim 6, wherein the lipid is selected from the group consisting of: a glycerophosphethanolamine, a glycerophosphocholine, a triacylglycerol, a phosphosphingolipid, a glycerophosphoinositol, and a glycerophosphoserine.

8. The pharmaceutical composition of claim 6, wherein the lipid is selected from the group consisting of: a phosphatidic acid, a phosphatidylcholine, a phosphatidylethanolamine, a phosphatidylinositol, and a phosphatidylserine.

9. The pharmaceutical composition of claim 6, wherein the lipid is a phosphatidic acid.

10. The pharmaceutical composition of claim 6, wherein the lipid is a phosphatidic acid, or a mixture of phosphatidic acids, each phosphatidic acid having the chemical structure: 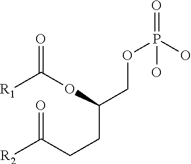
where each R1 and R2 is independently selected from saturated or unsaturated C16 to C22 phosphatidic acid.

11. A method of inhibiting and/or reversing growth restriction of a fetus of a subject pregnant with the fetus, wherein the fetus is exposed to ethanol in utero, comprising:
administering, to the pregnant subject, a therapeutically effective amount of a lipid which is downregulated in a pregnant subject exposed to ethanol during pregnancy.

12. The method of claim 11, wherein the lipid is selected from the group consisting of: a glycerophosphethanolamine, a glycerophosphocholine, a triacylglycerol, a phosphosphingolipid, a glycerophosphoinositol, and a glycerophosphoserine.

13. The method of claim 11, wherein the lipid is selected from the group consisting of: a phosphatidic acid, a phosphatidylcholine, a phosphatidylethanolamine, a phosphatidylinositol, and a phosphatidylserine.

14. The method of claim 11, wherein the lipid is a phosphatidic acid.

15. The method of claim 11, wherein the lipid is a phosphatidic acid, or a mixture of phosphatidic acids, each phosphatidic acid having the chemical structure: 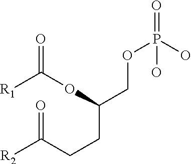
where each R1 and R2 is independently selected from saturated or unsaturated C16 to C22 phosphatidic acid.

16. A method of inhibiting and/or reversing blood vessel constriction in a pregnant subject exposed to ethanol during pregnancy, comprising:
administering, to the pregnant subject, a therapeutically effective amount of a lipid which is downregulated in a pregnant subject exposed to ethanol during pregnancy.

17. The method of claim 16, wherein the lipid is selected from the group consisting of: a glycerophosphethanolamine, a glycerophosphocholine, a triacylglycerol, a phosphosphingolipid, a glycerophosphoinositol, and a glycerophosphoserine.

18. The method of claim 16 wherein the lipid is selected from the group consisting of: a phosphatidic acid, a phosphatidylcholine, a phosphatidylethanolamine, a phosphatidylinositol, and a phosphatidylserine.

19. The method of claim 16, wherein the lipid is a phosphatidic acid.

20. The method of claim 16, wherein the lipid is a phosphatidic acid, or a mixture of phosphatidic acids, each phosphatidic acid having the chemical structure: 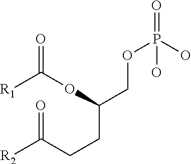
where each R1 and R2 is independently selected from saturated or unsaturated C16 to C22 phosphatidic acid.
